(19) United States
(12) Patent Application Publication
(10) Pub. No.: US 2018/0125386 A1
(43) Pub. Date: May 10, 2018

(54) BRAINWAVE SENSOR UNIT AND BRAINWAVE MEASUREMENT DEVICE USING SAME

(71) Applicant: Samsung Electronics Co., Ltd., Suwon-si, Gyeonggi-do (KR)

(72) Inventors: Se-hoon LIM, Suwon-si (KR); Hee-jae JO, Suwon-si (KR); Jun-hyung PARK, Seoul (KR); Jang-beom YANG, Suwon-si (KR); Jae-min JUNG, Suwon-si (KR); Jun-ho KOH, Suwon-si (KR); Chang-hyun LEE, Suwon-si (KR); Yong-hyun LIM, Suwon-si (KR)

(21) Appl. No.: 15/577,752

(22) PCT Filed: Apr. 22, 2016
(86) PCT No.: PCT/KR2016/004213

§ 371 Date: Nov. 28, 2017

(30) Foreign Application Priority Data
May 28, 2015 (KR) 10-2015-0074805

(51) Int. Cl. A61B 5/0478; G06F 3/01; A61B 5/00

(52) U.S. Cl. CPC A61B 5/0478; A61B 5/048; A61B 5/7207; G06F 3/015

(57) ABSTRACT

Disclosed are a brainwave sensor unit and a brainwave measurement apparatus using the same. The brainwave sensor unit includes first and second contact electrodes located on a supporter, the first contact electrode obtains a brainwave signal from a living body, and the second contact electrode is spaced apart and electrically insulated from the first contact electrode and is grounded.

### CROSS-REFERENCE TO RELATED APPLICATIONS AND CLAIM OF PRIORITY

This application is a National Stage of International Application No. PCT/KR2016/004213, filed Apr. 22, 2016, which claims the benefit of Korean Application No. 10-2015-0074805, filed May 28, 2015, the contents of which are incorporated herein by reference.

### TECHNICAL FIELD

The present disclosure relates to a brainwave sensor unit and a brainwave measurement apparatus using the same, and more particularly, to an electrode structure of a brainwave sensor unit and a circuit of a brainwave measurement apparatus capable of compensating motion artifact.

### BACKGROUND

A brainwave, that is, an electroencephalography (EEG) signal is an electrical biosignal recorded by measuring potential variations based on activity of the brain from the head of a living body. The brainwave signal is provided in the form of complicated waves having various potential variations, and the waves are analyzed in terms of amplitude and frequency. A method of obtaining the brainwave signal includes an invasive method for directly inserting electrodes into the scalp and the skull, and a non-invasive method for attaching electrodes onto the scalp. The invasive method may accurately measure the brainwave signal, but may cause infection during insertion and measurement and cause pain in a surgical procedure, and thus may not be easily used to measure the brainwave signal. Therefore, the non-invasive method is commonly used to measure the brainwave signal, and a representative example of the non-invasive method is a wet method using an electrolyte such as gel or saline solution. However, using the wet method, a sensor attaching procedure is inconvenient and the head or hair gets wet with the gel or saline solution. In addition, when the gel is hardened or the saline solution is evaporated, distortion of the signal occurs.

To solve the above problems, research is being actively conducted on a dry method using neither gel nor saline solution. The dry method should obtain a biosignal without using an electrolyte, and thus uses a conductor such as gold or silver for electrodes. In the dry method, the biosignal is measured while sensor electrodes are attached to physically contact the head of a user. However, if the user moves, motion slightly occurs between the sensor electrodes and a living body and thus impedance variations unavoidably occur. Due to the motion between the sensor electrodes and the scalp, the strength of contact may be changed, the strength of contact may be maintained but contact surfaces may slide aside, or the strength of contact may be changed and the contact surfaces may slide aside. As described above, impedance variations occur due to motion of the contact surfaces between sensor electrodes and the scalp. The impedance variations serve as noise (motion artifact) to a biosignal collected by a biosignal measurement apparatus and thus the measured signal has waveform distortion.

The signal distortion due to the impedance variations may be compensated by estimating motion artifact and removing the estimated motion artifact from the measured biosignal. Known methods of estimating the motion artifact include an impedance method, a half cell potential method, an optical method, and a method using an acceleration sensor. The impedance method is a method of differential-measuring difference information of impedance components of motion artifact by applying a certain voltage Vc or current Ic to a living body when a biosignal is measured, measuring motion artifact occurring when the biosignal is measured, and compensating the motion artifact. However, in the above method, an additional electrode is required to apply the voltage Vc or the current Ic to the living body to measure the motion artifact. If motion occurs in this electrode due to motion of the living body, this serves as an additional noise signal to an electrode for measuring the biosignal, thereby increasing difficulties in signal analysis.

### SUMMARY

Provided are a brainwave sensor unit having an improved electrode structure and including a circuit for compensating signal distortion, to reduce motion noise (motion artifact) occurring due to motion of a user when a brainwave signal is measured by using a dry sensor in daily life, and a brainwave measurement apparatus using the brainwave sensor units.

According to an aspect of an embodiment, a brainwave sensor unit includes first and second contact electrodes having a tapered shape to contact a living body, a signal line configured to transmit a brainwave signal obtained by the first contact electrode, to a signal processor, a ground line configured to ground the second contact electrode, and a supporter configured to separate and electrically insulate the first and second contact electrodes from each other. The brainwave signal obtained by the first contact electrode includes not only BRAINWAVE information but also motion artifact as will be described below, and the signal processor may remove the motion artifact included in the brainwave signal. As will be described below, the signal processor is a circuit included in a main body of a brainwave measurement apparatus to process brainwave signals obtained by the brainwave sensor unit.

A support surface of the supporter, which supports the first and second contact electrodes, may include a flat, bent, or curved surface.

The first and second contact first contact electrode electrodes may protrude from the support surface of the supporter. For example, the first and second contact electrodes may be made of a flexible material to protrude from the support surface of the supporter. In this case, a distance between the first and second contact electrodes may be determined based on a height and a base width of the first and second contact electrodes. For example, when the height and the base width of the first and second contact electrodes with respect to the support surface of the supporter are denoted by h and w, respectively, a minimum distance dₘᵢₙ between the first and second contact electrodes may satisfy dₘᵢₙ=h/2+w.

A maximum distance between the first and second contact electrodes may satisfy 80% of a correlation of the brainwave signal measured by the brainwave sensor unit with respect to a brainwave signal measured by a patch-type BRAINWAVE sensor.

A distance between the first and second contact electrodes may be between 0.5 mm and 5 mm.

The number of the first contact electrodes may be at least one. Likewise, the number of the second contact electrodes may be at least one. In this case, the number of the first contact electrodes may be equal to or greater than the number of the second contact electrodes.

The first and second contact electrodes may be provided in pairs located adjacent to each other.

The support surface of the supporter may include a first region and a second region, and a plurality of the first contact electrodes may be provided on the first region and a plurality of the second contact electrodes may be provided on the second region. Herein, the first and second regions do not overlap each other on the support surface. For example, the second region may include a center region of the support surface, and the first region may include an edge region of the support surface.

The first and second contact electrodes may include at least three contact electrodes protruding from the support surface of the supporter, and ends of the at least three contact electrodes may not be located on the same plane. For example, the ends of the at least three contact electrodes may circumscribe a circle having a radius R.

A height of the first contact electrode may be different from a height of the second contact electrode with respect to the support surface of the supporter.

A height of the first contact electrode may be equal to a height of the second contact electrode with respect to the support surface of the supporter, and the support surface of the supporter may be bent or curved. For example, the support surface of the supporter may include a curved surface which circumscribes a circle having a radius R.

A material of the first and second contact electrodes may include one of conductive silicone, conductive rubber, and metal.

The first and second contact electrodes may have one of a cylinder shape, a cone shape, a quadrangular pyramid shape, a rectangular prism shape, a funnel shape, and a curved funnel shape.

The first and second contact electrodes may be made of the same material and may have the same shape.

According to an aspect of another embodiment, a brainwave measurement apparatus includes a first brainwave sensor unit including a first contact electrode configured to obtain a first brainwave signal from a first location of a living body, a second contact electrode spaced apart and electrically insulated from the first contact electrode, a first signal line configured to transmit the first brainwave signal obtained by the first contact electrode, to a signal processor, a first ground line configured to ground the second contact electrode, and a first supporter configured to support the first and second contact electrodes, a second brainwave sensor unit including a third contact electrode configured to obtain a second brainwave signal from a second location of the living body, a fourth contact electrode spaced apart and electrically insulated from the third contact electrode, a second signal line configured to transmit a second brainwave signal obtained by the third contact electrode, to the signal processor, a second ground line configured to ground the fourth contact electrode, and a second supporter configured to support the third and fourth contact electrodes, and the signal processor configured to process the first and second brainwave signals obtained by the first and second brainwave sensor units. The first and second locations of the living body are spaced apart from each other. The first and second locations of the living body may include the scalp, ears (outer ears), back parts of ears, forehead, temples, etc. of a user.

The signal processor may include a first voltage divider connected to the first signal line of the first brainwave sensor unit and a voltage source to output a first voltage signal voltage-divided from the first brainwave signal received from the first brainwave sensor unit, and the voltage source, a second voltage divider connected to the second signal line of the second brainwave sensor unit and the voltage source to output a second voltage signal voltage-divided from the second brainwave signal received from the second brainwave sensor unit, and the voltage source, and a differential amplifier connected to the first signal line of the first brainwave sensor unit and the second signal line of the second brainwave sensor unit and configured to amplify a difference value between the first and second voltage signals.

The signal processor may extract a first impedance between the first contact electrode of the first brainwave sensor unit and the living body from the first voltage signal output from the first voltage divider, extract a second impedance between the third contact electrode of the second brainwave sensor unit and the living body from the second voltage signal output from the second voltage divider, and remove motion artifact from the first and second brainwave signals based on the first and second impedances.

A first distance between the first and second contact electrodes of the first brainwave sensor unit may be equal to a second distance between the third and fourth contact electrodes of the second brainwave sensor unit.

A circuit of the brainwave measurement apparatus may further include a communication unit configured to communicate with an external device, an output unit configured to output an alert, and a controller configured to determine an emergency level of a user based on a brainwave signal processed by the signal processor, and to control the output unit to output information corresponding to the determined emergency level or control the communication unit to transmit information about the determined emergency level to the external device. The output unit may include a speaker, a lamp, or a display. For example, a state of the user determined by the controller may include an emergency situation. In other words, the controller may predict an emergency situation or determine that an emergency situation has occurred, based on the brainwave signal obtained by the sensor. When the state of the user determined by the controller corresponds to an emergency situation, the controller may control to transmit information about the emergency situation of the user to the external device, or control to output an alert. The brainwave measurement apparatus may further include a memory configured to store a risk level evaluation model for evaluating a first risk level and a second risk level higher than the first risk level, based on the brainwave signal, and the controller may control the output unit to output the alert if the emergency level of the user corresponds to the first risk level, or control the communication unit to transmit the information about the emergency level of the user to the external device if the emergency level of the user corresponds to the second risk level. In some cases, the controller may control the output unit to output the alert if the emergency level of the user corresponds to the second risk level, or control the communication unit to transmit the information about the emergency level of the user to the external device if the emergency level of the user corresponds to the first risk level.

The emergency level of the user may include the first risk level and the second risk level higher than the first risk level, and the controller may control the communication unit to transmit the brainwave signal processed by the signal processor, to an external computer device and to receive information about the emergency level of the user generated by processing the brainwave signal, and control the output unit to output the alert if the emergency level of the user received from the computer device corresponds to the first risk level, or control the communication unit to transmit the information about the emergency level of the user to the external device if the emergency level of the user received from the computer device corresponds to the second risk level. In some cases, the controller may control the output unit to output the alert if the emergency level of the user received from the computer device corresponds to the second risk level, or control the communication unit to transmit the information about the emergency level of the user to the external device if the emergency level of the user received from the computer device corresponds to the first risk level.

According to an aspect of another embodiment, a brainwave measurement system includes the above-described brainwave measurement apparatus, and a brainwave measurement processing apparatus configured to receive a brainwave signal from the brainwave measurement apparatus and to process the brainwave signal.

The brainwave processing apparatus may include a mobile device. The mobile device may include a communication unit configured to communicate with the brainwave measurement apparatus, an output unit configured to output an alert, a memory configured to store information related to brainwave processing, a signal processor configured to process the brainwave signal received from the brainwave measurement apparatus with reference to the memory, and a controller configured to control the output unit based on the brainwave signal processed by the signal processor.

For example, the mobile device may include a communication unit configured to communicate with the brainwave measurement apparatus and an external device, an output unit configured to output an alert, and a controller configured to determine an emergency level of a user based on the brainwave signal received from the brainwave measurement apparatus, and to control the output unit to output an alert corresponding to the determined emergency level or control the communication unit to transmit information about the determined emergency level to the external device.

The mobile device may further include a memory configured to store a risk level evaluation model for evaluating a first risk level and a second risk level higher than the first risk level, based on the brainwave signal, and the controller may control the output unit to output the alert if the emergency level of the user corresponds to the first risk level, or control the communication unit to transmit the information about the emergency level of the user to the external device if the emergency level of the user corresponds to the second risk level. In some cases, the controller may control the output unit to output the alert if the emergency level of the user corresponds to the second risk level, or control the communication unit to transmit the information about the emergency level of the user to the external device if the emergency level of the user corresponds to the first risk level.

The mobile device may include a communication unit configured to communicate with the brainwave measurement apparatus and a computer device, an output unit configured to output an alert, and a controller configured to transmit the brainwave signal received from the brainwave measurement apparatus, to the computer device, to receive information about a state of the user generated by processing the brainwave signal, from the computer device, and control the output unit and the communication unit based on the received information about the state of the user. For example, the computer device may generate information about an emergency level of the user by processing the brainwave signal. For example, the emergency level of the user may include a relative low first risk level and a relative high second risk level. The controller of the mobile device may transmit the brainwave signal received from the brainwave measurement apparatus, to the computer device and receive the information about the emergency level of the user generated by processing the brainwave signal, from the computer device through the communication unit, and control the output unit to output the alert if the emergency level of the user received from the computer device corresponds to the first risk level, or control the communication unit to transmit information about an emergency situation of the user to the external device if the emergency level of the user received from the computer device corresponds to the second risk level. The computer device and the external device may be configured as the same device or different devices. For example, the computer device may include a server of a remote medical service provider, and the external device may include a server of an emergency center, a server of a hospital where the user usually goes, a phone of a primary care doctor of the user, or a phone of a guardian of the user. The information about the emergency situation of the user may be transmitted to the external device directly by the communication unit of the mobile device. Otherwise, the computer device may be instructed to transmit the information about the emergency situation of the user to the external device or the information about the emergency situation of the user may be automatically transmitted to the external device based on a scenario stored in a memory of the computer device.

The mobile device may include a mobile phone, a smartphone, a tablet computer, a personal digital assistant (PDA), or a laptop computer. The mobile device may transmit the processed brainwave information to a computer device connected via a network. In addition, the mobile device may include at least one of a location tracking device for tracking a location of a living body, an acceleration sensor for measuring acceleration of the living body, and a motion sensor for measuring motion of the living body, and transmit information about at least one of the location and motion of the living body to the computer device.

The brainwave processing apparatus may include a computer device configured to communicate with the brainwave measurement apparatus. The computer device may include a communication unit configured to directly communicate with the brainwave measurement apparatus to receive a brainwave signal from the brainwave measurement apparatus, a memory configured to store a risk level evaluation model for evaluating a first risk level and a second risk level higher than the first risk level, based on the brainwave signal, and a controller configured to control the output unit to transmit a warning message to the brainwave measurement apparatus if an emergency level of a user corresponds to the first risk level, or control the communication unit to transmit information about an emergency situation of the user to the external device if the emergency level of the user corresponds to the second risk level. The computer device may include a server of a remote medical service provider, a server of a hospital where the user usually goes, or a personal computer of the user. The external device may include a server of an emergency center, a server of a hospital where the user usually goes, a phone of a primary care doctor of the user, or a phone of a guardian of the user.

The output unit configured to output the brainwave information processed by the brainwave processing apparatus may be embedded in or connected to the brainwave measurement apparatus or the mobile device. The output unit may include a speaker, a vibration module, a lamp, or a display. For example, the brainwave measurement apparatus may include the vibration module to output an alert as vibration. As another example, the mobile device may include a speaker, a vibration module, and a display, and output an alert as alert sound, vibration, a warning message, etc.

The brainwave processing apparatus may include at least one of an emergency situation prediction module configured to predict an emergency situation or determine that an emergency situation has occurred, based on the brainwave information, and a living body intention inference module configured to infer an intention of a living body based on the brainwave information.

For example, the brainwave processing apparatus may predict an emergency situation or determine that an emergency situation has occurred, based on the brainwave information, and transmit an alert to an output device when the emergency situation is predicted or has occurred, and the output device may output an alert. The brainwave information may include at least one of electroencephalography (EEG), electrocardiogram (ECG), electromyogram (EMG), electroneurogram (ENoG), and electrooculogram (EOG) signals, and the brainwave processing apparatus may infer an intention or state of the living body based on the brainwave information.

As another example, the brainwave processing apparatus may transmit information about the inferred intention or state to the output device, and the output device may output the information about the inferred intention or state. The brainwave processing apparatus may generate control information based on the information about the inferred intention or state, and transmit the control information to an electronic device.

The brainwave measurement apparatus may further include a sensor configured to measure at least one of a body temperature, a heart rate, nodding, blinking, and tossing and turning of the living body. At least one of a location tracking device for tracking a location of a living body, an acceleration sensor for measuring acceleration of the living body, and a motion sensor for measuring motion of the living body may be further provided. The additional sensor may be included in the brainwave measurement apparatus or another electronic device.

According to an aspect of another embodiment, a brainwave processing method includes measuring a brainwave signal of a living body by using the above-described brainwave measurement apparatus, and generating information about the living body by processing the measured brainwave signal.

The method may further include predicting an emergency situation or determining whether an emergency situation has occurred, based on the information about the living body, and outputting an alert to a user when the emergency situation is predicted or has occurred.

The generating of the information about the living body may include inferring an intention or state of the living body, based on the brainwave signal. The measuring of the brainwave signal of the living body may further include measuring at least one of electrocardiogram (ECG), electromyogram (EMG), electroneurogram (ENoG), and electrooculogram (EOG) signals of the living body. The measuring of the brainwave signal of the living body may further include measuring at least one of a body temperature, a heart rate, nodding, blinking, and tossing and turning of the living body. The method may further include transmitting information about the inferred intention or state of the living body to the user.

The method may further include tracking a location of the living body, and the information transmitted to the user may include information about the location of the living body.

The user may include at least one of the living body, a guardian of the living body, and a medical specialist.

A brainwave sensor unit according to an embodiment may be easily used in daily life because electrodes independently measure motion artifact and thus an electrode having motion artifact does not influence the other electrode.

In the brainwave sensor unit according to an embodiment, the size and occurrence of motion artifact which varies as time passes may be measured and compensated in real time and thus signal analysis may be easily performed.

### BRIEF DESCRIPTION OF THE DRAWINGS

FIG. 1 is a schematic view of a brainwave measurement apparatus according to an embodiment.

FIG. 2A is a perspective view of a brainwave sensor unit of the brainwave measurement apparatus of FIG. 1.

FIG. 2B is a cross-sectional view of the brainwave sensor unit of FIG. 2A taken along line I-I′.

FIG. 3 is a graph showing a correlation based on the distance between first and second contact electrodes of a brainwave sensor unit.

FIGS. 4A to 4D are brainwave signal graphs showing correlations based on the distance between the first and second contact electrodes of the brainwave sensor unit.

FIG. 5 is a block diagram of the brainwave measurement apparatus of FIG. 1.

FIGS. 6A and 6B are equivalent circuit diagrams for describing voltage division from brainwave signals and a voltage source.

FIGS. 7A and 7B show other examples of contact electrode arrangement of a brainwave sensor unit.

FIGS. 8A to 8D show examples of contact electrodes of a brainwave sensor unit.

FIG. 9 is a side cross-sectional view of a brainwave sensor unit according to another embodiment.

FIG. 10 is a view for describing heights of contact electrodes of the brainwave sensor unit of FIG. 9.

FIGS. 11A and 11B show modified examples of the brainwave sensor unit of FIG. 9.

FIG. 12 is a side cross-sectional view of a brainwave sensor unit according to still another embodiment.

FIG. 13 is a view for describing heights of contact electrodes of the brainwave sensor unit of FIG. 12.

FIGS. 14A and 14B show modified examples of the brainwave sensor unit of FIG. 12.

FIGS. 15A to 15C show other modified examples of the brainwave sensor unit of FIG. 13.

FIG. 16 is a block diagram of a brainwave measurement apparatus according to another embodiment.

FIG. 17 is a block diagram of a brainwave measurement system according to an embodiment.

FIG. 18 is a detailed block diagram of the brainwave measurement system of FIG. 17.

FIG. 19 shows an example of a controller and a memory of a mobile device in the brainwave measurement system of FIG. 18.

FIG. 20 shows a brainwave learning process for diagnosing a stroke.

FIG. 21 shows a stroke evaluation process.

FIG. 22 is a flowchart of a risk level determination process based on stroke evaluation.

FIG. 23 shows another example of the controller and the memory of the mobile device in the brainwave measurement system of FIG. 18.

FIG. 24 is a block diagram of a brainwave measurement system according to another embodiment.

FIG. 25 is a detailed block diagram of a computer device in the brainwave measurement system of FIG. 24.

FIG. 26 is a block diagram of a brainwave measurement system according to still another embodiment.

FIG. 27 is a block diagram of a brainwave measurement system according to still another embodiment.

### DETAILED DESCRIPTION

The present disclosure and methods of accomplishing the same may be understood more readily by reference to the following detailed description of embodiments and the accompanying drawings. However, the present disclosure may be embodied in many different forms, and should not be construed as being limited to the embodiments set forth herein. Rather, these embodiments are provided so that this disclosure will be through and complete and will fully convey the concept of the disclosure to those skilled in the art, and the present disclosure will only be defined by the appended claims. In the drawings, like reference numerals denote like elements and the sizes or thicknesses of elements may be exaggerated for clarity of explanation.

The terminology used herein will be described briefly, and the present disclosure will be described in detail.

The terminology used herein is defined in consideration of the function of corresponding components used in the present disclosure and may be varied according to users, operator's intention, or practices. In addition, an arbitrary defined terminology may be used in a specific case and will be described in detail in a corresponding description paragraph. Therefore, the terminology used herein is for the purpose of describing particular embodiments only and is not intended to be limiting of the disclosure.

Throughout the specification, unless explicitly described to the contrary, the word “comprise” and variations such as “comprises” or “comprising” will be understood to imply the inclusion of stated elements but not the exclusion of any other elements.

Hereinafter, the present disclosure will now be described more fully with reference to the accompanying drawings, in which embodiments are shown such that one of ordinary skill in the art may easily understand the disclosure. Details that are not related to description of the disclosure will be omitted for clarity of explanation.

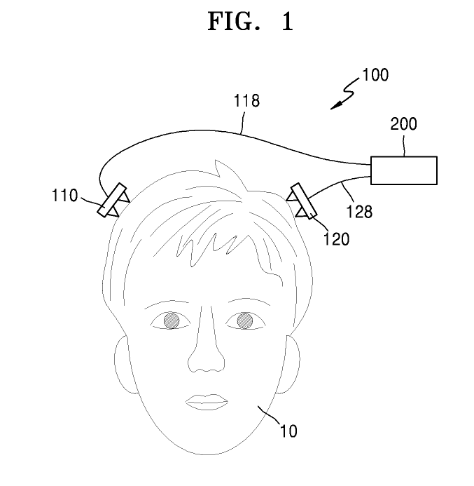

FIG. 1 is a schematic view of a brainwave measurement apparatus according to an embodiment.

Referring to FIG. 1, the brainwave measurement apparatus according to the current embodiment includes a sensor 100 and a signal processor 200. The sensor 100 includes first and second brainwave sensor units 110 and 120. The first and second brainwave sensor units 110 and 120 obtain first and second brainwave signals from different locations of a living body 10. Parts of the living body 10 to which the first and second brainwave sensor units 110 and 120 are attachable include the scalp, ears (outer ears), back parts of ears, forehead, temples, etc. of a user. The first and second brainwave sensor units 110 and 120 may be supported by a frame (not shown) and may be fixed or attached onto the living body 10. The signal processor 200 may be located in the frame which supports the first and second brainwave sensor units 110 and 120, or in a housing. The housing which accommodates the signal processor 200 may have, for example, a shape of an accessory worn by a user at ordinary times or a shape attached to the accessory. The brainwave signals obtained by the first and second brainwave sensor units 110 and 120 are transmitted to the signal processor 200 through cables 118 and 128 and are processed.

The first and second brainwave sensor units 110 and 120 have the same structure. In other words, the first and second brainwave sensor units 110 and 120 may have the same shape and the same size and may be made of the same material. Hereinafter, for convenience of explanation, the first brainwave sensor unit 110 will be described representatively and a description of the second brainwave sensor unit 120 will not be provided.

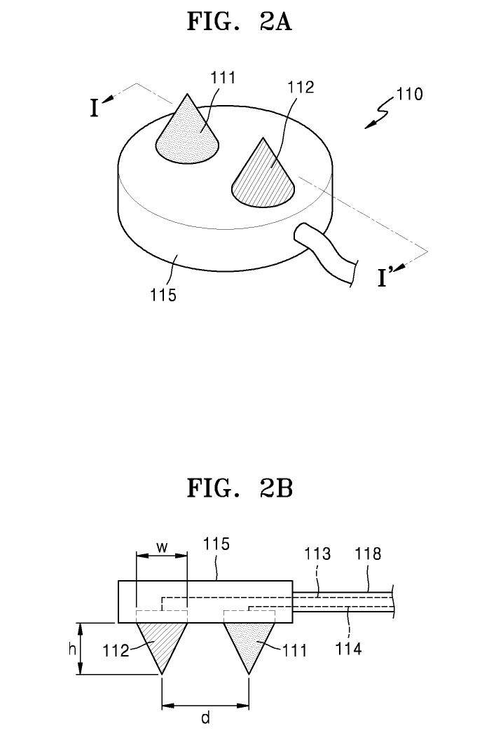

FIG. 2A is a perspective view of the first brainwave sensor unit 110, and FIG. 2B is a cross-sectional view of the first brainwave sensor unit 110 taken along line I-I′.

Referring to FIGS. 2A and 2B, the first brainwave sensor unit 110 includes a first contact electrode 111, and a second contact electrode 112 spaced apart from the first contact electrode 111. Herein, the fact that the first and second contact electrodes 111 and 112 are spaced apart from each other means that the first and second contact electrodes 111 and 112 are physically separated from each other. The first and second contact electrodes 111 and 112 are supported by a supporter 115. The first brainwave sensor unit 110 is connected to the signal processor 200 through the cable 118. The cable 118 may include a first signal line 113 and a first ground line 114.

The first and second contact electrodes 111 and 112 refer to unit electrodes contacting the living body. The first and second contact electrodes 111 and 112 have the same shape and the same size and are made of the same material. For example, as illustrated in FIGS. 2A and 2B, the first and second contact electrodes 111 and 112 may have a tapered shape, e.g., a cone shape, protruding from a support surface of the supporter 115, and may be made of a flexible and conductive material. The term flexible means that the material has flexibility and thus is bent due to external force. The flexible and conductive material may be, for example, conductive polymer such as conductive silicone or conductive rubber. The first and second contact electrodes 111 and 112 may be made of the conductive polymer or flexible and conductive synthetic resin. The first and second contact electrodes 111 and 112 may be made of rigid and conductive synthetic resin. Otherwise, the first and second contact electrodes 111 and 112 may be made of a conductive metal material or another rigid material. The first and second contact electrodes 111 and 112 may be understood as non-invasive dry electrodes. The cone shape of the first and second contact electrodes 111 and 112 is an example of electrode structures protruding from the support surface of the supporter 115. Herein, the support surface refers to a surface of the supporter 115, which supports the first and second contact electrodes 111 and 112. In other words, the support surface refers to a surface of the supporter 115, on which the first and second contact electrodes 111 and 112 are located.

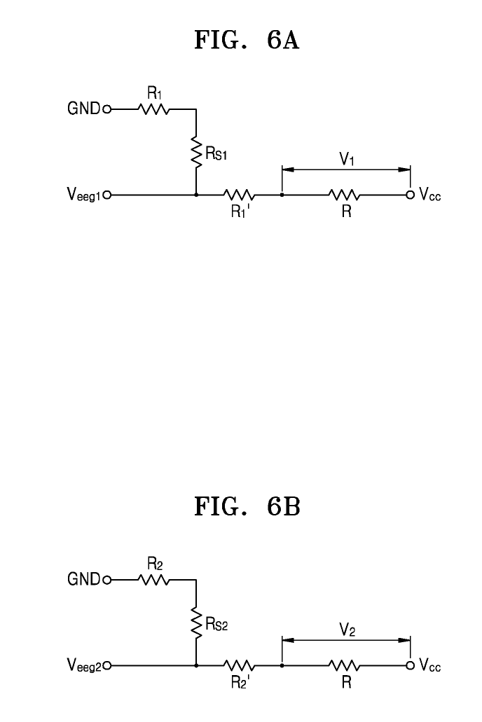

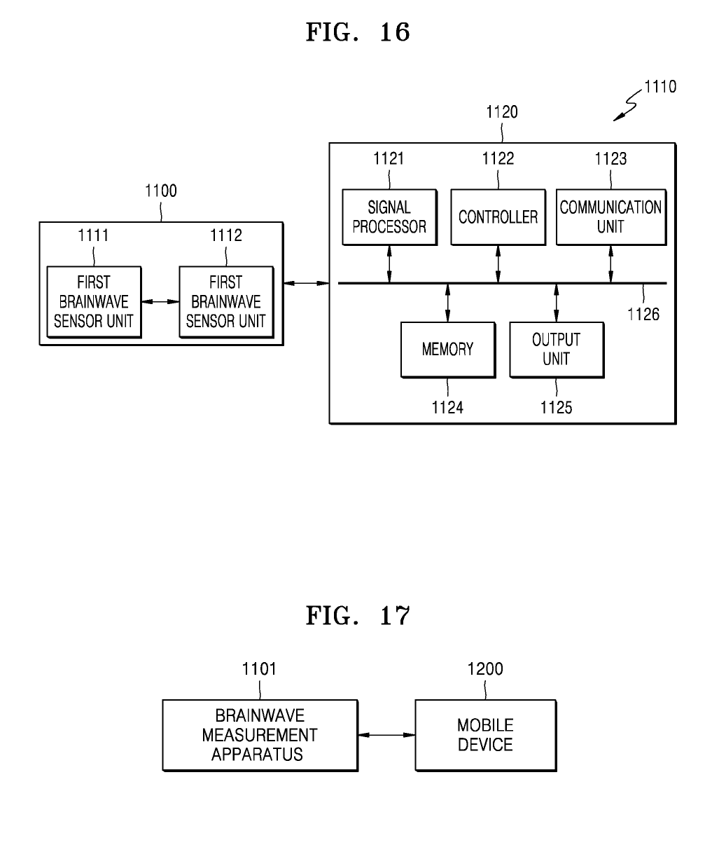

The first contact electrode 111 obtains a brainwave signal from the living body 10. The brainwave signal obtained by the first contact electrode 111 is transmitted to the signal processor 200 through the first signal line 113. The second contact electrode 112 is electrically insulated from the first contact electrode 111 and is connected to the ground of a circuit 1120 (see FIG. 16). The second contact electrode 112 may be connected to the ground through the first ground line 114. Herein, the fact that the first and second contact electrodes 111 and 112 are insulated from each other means that the first and second contact electrodes 111 and 112 are not connected to each other by a conductor. The first and second contact electrodes 111 and 112 are located adjacent to the skin of the living body to measure the brainwave signal, and a skin resistance proportional to skin contact resistances R₁ and R₁′ (see FIG. 6B) of the first and second contact electrodes 111 and 112 and the distance between the first and second contact electrodes 111 and 112 will be present between the first and second contact electrodes 111 and 112. Although a sensor of a conventional brainwave measurement apparatus includes a ground electrode separately from a brainwave electrode, in the sensor 100 according to the current embodiment, the first brainwave sensor unit 110 includes the second contact electrode 112 corresponding to a ground electrode and thus a ground sensor electrode is not additionally required.

The supporter 115 separates and electrically insulates the first and second contact electrodes 111 and 112 from each other. The supporter 115 may be made of a non-conductive material. For example, the supporter 115 may be made of non-conductive synthetic resin. Lines of the first and second contact electrodes 111 and 112 may be provided in the supporter 115 or a surface of the supporter 115 opposite to the support surface. The lines provided in the supporter 115 (i.e., the first signal line 113 and the first ground line 114) extend out of the supporter 115 along the cable 118. The supporter 115 may have rigidity to maintain the distance between the first and second contact electrodes 111 and 112.

The first and second contact electrodes 111 and 112 are insulated from each other. The first and second contact electrodes 111 and 112 may be provided adjacent to each other. However, to ensure insulation between the first and second contact electrodes 111 and 112, the minimum distance between the first and second contact electrodes 111 and 112 may be limited depending on the material and shape of the first and second contact electrodes 111 and 112. For example, since the first and second contact electrodes 111 and 112 may be made of a flexible material as described above, ends of the first and second contact electrodes 111 and 112 may be bent when contacting the living body 10 and thus a short circuit may be caused if a distance d between the first and second contact electrodes 111 and 112 is excessively small. Therefore, considering that the ends of the first and second contact electrodes 111 and 112 are bent, the first and second contact electrodes 111 and 112 may be spaced apart from each other by more than a minimum distance dₘᵢₙ. For example, when the first and second contact electrodes 111 and 112 have a flexible cone shape and a height and a base width of the first and second contact electrodes 111 and 112 are denoted by h and w, respectively, the minimum distance dₘᵢₙ between the first and second contact electrodes 111 and 112 may satisfy Equation 1.

$${d}_{min} = \frac{h}{2} + w \qquad Equation 1$$

As shown in Equation 1, the minimum distance dₘᵢₙ between the first and second contact electrodes 111 and 112 may be increase in proportion to the size of the first and second contact electrodes 111 and 112. For example, when the height h and the base width w of the first and second contact electrodes 111 and 112 are 0.3 mm and 0.2 mm, respectively, the minimum distance between the first and second contact electrodes 111 and 112 may be 0.5 mm. When the height h and the base width w of the first and second contact electrodes 111 and 112 are 1 mm and 0.25 mm, respectively, the minimum distance between the first and second contact electrodes 111 and 112 may be 1 mm. Otherwise, when the height h and the base width w of the first and second contact electrodes 111 and 112 are 2.5 mm and 3 mm, respectively, the minimum distance between the first and second contact electrodes 111 and 112 may be 2.75 mm.

If the first and second contact electrodes 111 and 112 are made of a conductive and rigid material such as metal, the minimum distance between the first and second contact electrodes 111 and 112 may be determined within a range allowed in a manufacturing process thereof.

When the distance between the first and second contact electrodes 111 and 112 is increased, noise of the brainwave signal may also be increased. Therefore, to suppress the noise of the brainwave signal within a range processable by the signal processor 200, the distance between the first and second contact electrodes 111 and 112 should be limited. For example, a maximum distance dₘₐₓ between the first and second contact electrodes 111 and 112 may be determined based on an allowable maximum value of a correlation of a brainwave signal measured by the sensor 100 according to the current embodiment with respect to a brainwave signal measured by a patch-type brainwave sensor. The patch-type brainwave sensor includes electrodes in patches attached onto the living body 10, and is known as a non-invasive brainwave sensor structure relatively free from motion noise (motion artifact) caused by motion of a user near the electrodes, and other types of noise.

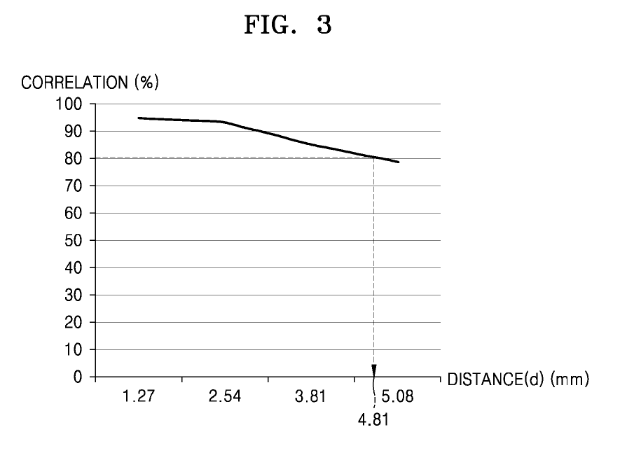

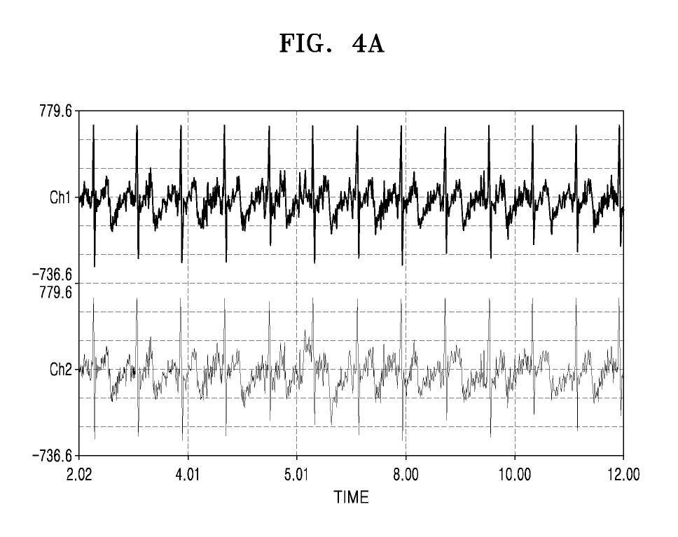

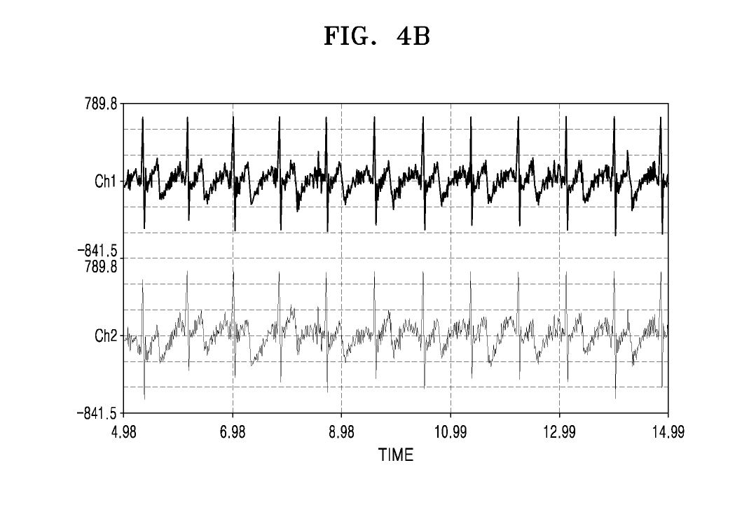

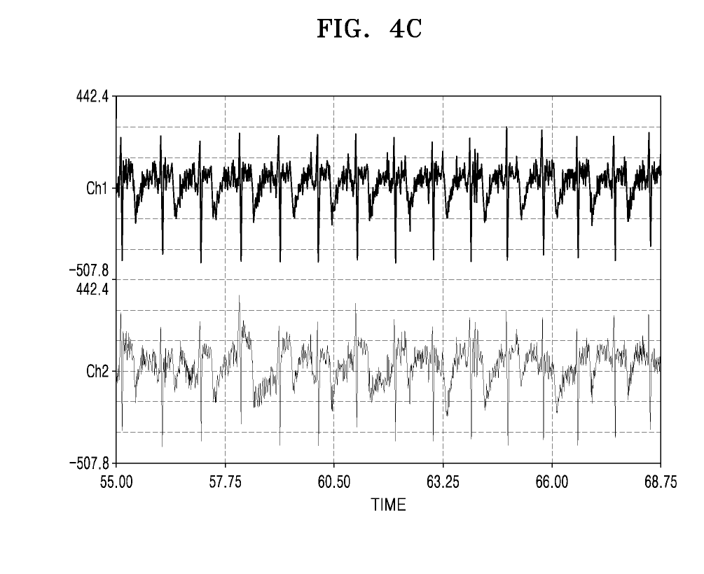

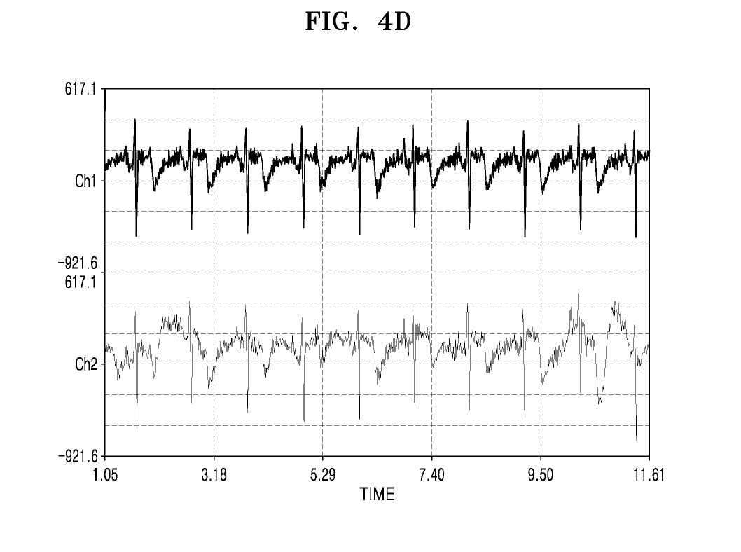

FIG. 3 is a graph showing a correlation based on a distance d between first and second contact electrodes of a brainwave sensor unit according to the current embodiment, and FIGS. 4A to 4D are brainwave signal graphs showing a brainwave signal obtained by the brainwave sensor unit according to the current embodiment (top) and a brainwave signal according to a comparative example (bottom) in a case when the distance d between the first and second contact electrodes of the brainwave sensor unit according to the current embodiment varies to 1.27 mm, 2.54 mm, 3.81 mm, and 5.08 mm. In FIGS. 3 and 4A to 4D, the sensor 100 according to the current embodiment has metallic button-type first and second contact electrodes, and the comparative example is a patch-type brainwave sensor.

Referring to FIG. 3, when the distance d between the first and second contact electrodes is increased, the correlation between the brainwave signal measured by the sensor 100 according to the current embodiment and the brainwave signal measured by the patch-type brainwave sensor is reduced. For example, as shown in FIG. 4A, when the distance d between the first and second contact electrodes of the brainwave sensor unit according to the current embodiment is 1.27 mm, the correlation between the brainwave signal measured by the sensor 100 according to the current embodiment and the brainwave signal measured by the patch-type brainwave sensor reaches 95.1%. As shown in FIGS. 4B, 4C, and 4D, when the distance d between the first and second contact electrodes of the brainwave sensor unit according to the current embodiment is increased to 2.54 mm, 3.81 mm, and 5.08 mm, the correlation between the brainwave signal measured by the sensor 100 according to the current embodiment and the brainwave signal measured by the patch-type brainwave sensor is reduced to 93.5%, 85.3%, and 78.9%.

It is known that a brainwave signal postprocessed by the signal processor 200 is easily analyzable if the brainwave signal has a correlation of about 85% with respect to the brainwave signal measured by the patch-type brainwave sensor. The brainwave signal obtained by the sensor 100 may be postprocessed by using an adaptive filter and thus signal performance may be additionally improved by about 5%. Therefore, the distance between the first and second contact electrodes 111 and 112 may be limited in such a manner that the correlation of the brainwave signal measured by the sensor 100 according to the current embodiment with respect to the brainwave signal measured by the patch-type brainwave sensor is at least 80%. Referring to FIG. 3, to ensure a correlation of 80% between the brainwave signal measured by the sensor 100 according to the current embodiment and the brainwave signal measured by the patch-type brainwave sensor, the distance d between the first and second contact electrodes should be about 4.81 mm. In other words, the maximum distance dₘₐₓ between the metallic button-type first and second contact electrodes may be 4.81 mm.

Depending on improvement of signal performance through postprocessing and development of brainwave signal analysis technology, an allowable maximum value of the correlation between the brainwave signal measured by the sensor 100 according to the current embodiment and the brainwave signal measured by the patch-type brainwave sensor may vary, and thus the maximum distance dₘₐₓ between the first and second contact electrodes 111 and 112 may also vary.

The maximum distance dₘₐₓ between the first and second contact electrodes may slightly vary depending on the material or shape of the first and second contact electrodes. For example, when the first and second contact electrodes 111 and 112 have a flexible cone shape as described above in relation to FIGS. 2A and 2B, the maximum distance dₘₐₓ may be, for example, 5 mm. In further consideration of the above-described minimum distance between the first and second contact electrodes 111 and 112, when each of the first and second contact electrodes 111 and 112 has a flexible cone shape and the height h and the base width w thereof are 1 mm and 0.25 mm, respectively, to easily analyze the brainwave signal, the distance between the first and second contact electrodes 111 and 112 may be determined within a range of 1 mm to 5 mm. As another example, when each of the first and second contact electrodes 111 and 112 has a flexible cone shape and the height h and the base width w thereof are 0.3 mm and 0.2 mm, respectively, to easily analyze the brainwave signal, the distance between the first and second contact electrodes 111 and 112 may be determined within a range of 0.5 mm to 5 mm.

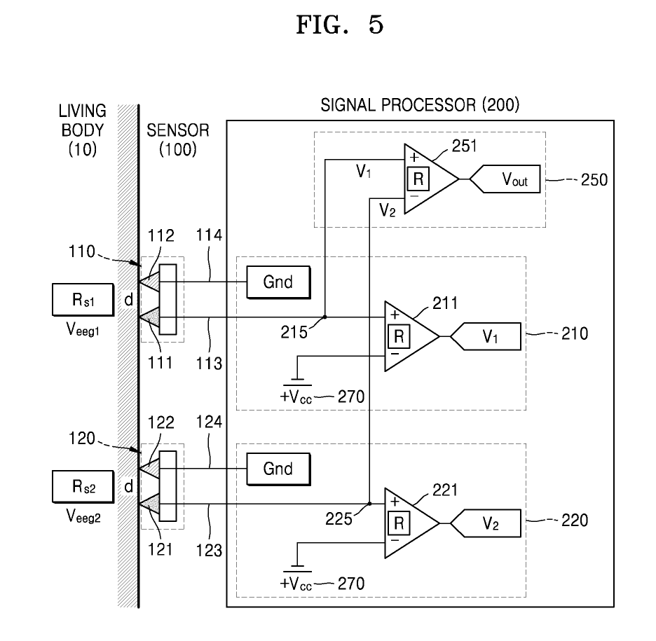

FIG. 5 is a block diagram of the brainwave measurement apparatus of FIG. 1, and FIGS. 6A and 6B are equivalent circuit diagrams for describing voltage division from brainwave signals and a voltage source.

Referring to FIG. 5, the sensor 100 includes the first and second brainwave sensor units 110 and 120 configured to measure brainwave signals from different locations of a living body, i.e., the living body 10. The first and second contact electrodes 111 and 112 of the first brainwave sensor unit 110 are spaced apart from each other by the distance d and contact a location of the living body 10. Likewise, third and fourth contact electrodes 121 and 122 of the second brainwave sensor unit 120 are spaced apart from each other by the distance d and contact another location of the living body 10. The first contact electrode 111 of the first brainwave sensor unit 110 obtains a first brainwave signal V_{eeg1} from the location of the living body 10 and transmits the same to the signal processor 200 through the first signal line 113. The third contact electrode 121 of the second brainwave sensor unit 120 obtains a second brainwave signal V_{eeg2} from the other location of the living body 10 and transmits the same to the signal processor 200 through a second signal line 123. The second contact electrode 112 of the first brainwave sensor unit 110 and the fourth contact electrode 122 of the second brainwave sensor unit 120 are connected to the ground GND through first and second ground lines 114 and 124.

The signal processor 200 includes first and second voltage dividers 210 and 220, and a differential amplifier 250.

The first and second voltage dividers 210 and 220 may include first and second operational amplifiers 211 and 221 having, for example, an internal impedance R. An inverting input terminal − of the first operational amplifier 211 may be connected to the first signal line 113 to receive the first brainwave signal V_{eeg1} input from the first brainwave sensor unit 110, and a non-inverting input terminal + thereof may be connected to a voltage source V_{cc}. The first operational amplifier 211 may output a first voltage V1. In this sense, the first voltage divider 210 may be understood as a first voltage meter. An inverting input terminal − of the second operational amplifier 221 may be connected to the second signal line 123 to receive the second brainwave signal V_{eeg2} input from the second brainwave sensor unit 120, and a non-inverting input terminal + thereof may be connected to the voltage source V_{cc}. The second operational amplifier 221 may output a second voltage V2. In this sense, the second voltage divider 220 may be understood as a second voltage meter.

The differential amplifier 250 may include an operational amplifier 251. Non-inverting and inverting input terminals + and − of the differential amplifier 250 are connected to the first and second signal lines 113 and 123. The differential amplifier 250 may output Vₒᵤₜ by amplifying, i.e., differential-amplifying, a difference value between the first and second voltages V1 and V2. Reference numeral 215 denotes a first node where the first signal line 113 is divided toward the non-inverting input terminal + of the first voltage divider 210 and the non-inverting input terminal + of the differential amplifier 250, and reference numeral 225 denotes a second node where the second signal line 123 is divided toward the non-inverting input terminal + of the second voltage divider 220 and the inverting input terminal − of the differential amplifier 250.

Referring to FIG. 6A, a contact impedance is present between the first contact electrode 111 of the first brainwave sensor unit 110 and the living body 10, and thus is approximated to a first contact resistance R₁. In addition, contact impedance between the second contact electrode 112 of the first brainwave sensor unit 110 and the living body 10 is approximated to a second contact resistance R₁′.

A first skin resistance Rₛ₁ due to the living body 10 is present between the first and second contact electrodes 111 and 112 of the first brainwave sensor unit 110. The first contact electrode 111 measures the first brainwave signal V_{eeg1}, and the second contact electrode 112 is grounded. The first voltage divider (operational amplifier) 210 has the internal impedance R. As such, the first voltage V1 of the first voltage divider (operational amplifier) 210 is given as a sum of voltage division by the voltage source V_{cc} and voltage division by the first brainwave signal V_{eeg1} as shown in Equation 2.

$${V}_{1} = \frac{R}{{R}_{c 1} + R} \times {V}_{cc} + \frac{R}{{R}_{1} + R} \times {V}_{eeg 1} ≅ \frac{R}{{R}_{c 1} + R} \times {V}_{cc} \qquad Equation 2$$

Herein, R_{c1} denotes a series combined resistance of the first and second contact resistances R₁ and R₁′ and the first skin resistance Rₛ₁ as shown in Equation 3.

R_{c1}=R₁+Rₛ₁+R₁′≅2R₁+Rₛ₁ Equation 3

Approximation in Equation 2 uses a fact that the first brainwave signal V_{eeg1} is a very small value compared to the voltage source Vₑₑ, and approximation in Equation 3 considers the first and second contact resistances R₁ and R₁′ to be the same by sufficiently reducing the distance d between the first and second contact electrodes 111 and 112 of the first brainwave sensor unit 110 as described above.

Referring to FIG. 6B, a third contact resistance R₂ is present between the third contact electrode 121 of the second brainwave sensor unit 120 and the living body 10, and a fourth contact resistance R₂′ is present between the fourth contact electrode 122 of the second brainwave sensor unit 120 and the living body 10. A second skin resistance Rₛ₂ due to the living body 10 is present between the third and fourth contact electrodes 121 and 122 of the second brainwave sensor unit 120. The third contact electrode 121 measures the second brainwave signal V_{eeg2}, and the fourth contact electrode 122 is grounded. The second voltage divider (operational amplifier) 220 has the internal impedance R. As such, the second voltage V2 of the second voltage divider (operational amplifier) 220 is given as a sum of voltage division by the voltage source V_{cc} and voltage division by the second brainwave signal V_{eeg2} as shown in Equation 4.

$${V}_{2} = \frac{R}{{R}_{c 2} + R} \times {V}_{cc} + \frac{R}{{R}_{2} + R} \times {V}_{eeg 2} ≅ \frac{R}{{R}_{c 2} + R} \times {V}_{cc} \qquad Equation 4$$

Herein, R_{c2} denotes a sum of the third and fourth contact resistances R₂ and R₂′ and the second skin resistance Rₛ₂ as shown in Equation 5.

R_{c2}=R₂+Rₛ₂+R₂′≅2R₂+Rₛ₂ Equation 5

Approximation in Equation 4 uses a fact that the second brainwave signal V_{eeg2} is a very small value compared to the voltage source V_{cc}, and approximation in Equation 5 considers the third and fourth contact resistances R₂ and R₂′ to be the same by sufficiently reducing the distance d between the third and fourth contact electrodes 121 and 122 of the second brainwave sensor unit 120 as described above.

To analyze the brainwave signals, Vₒᵤₜ given as shown in Equation 6 may be calculated by setting the second brainwave sensor unit 120 as a reference electrode and amplifying, i.e., differential-amplifying, the difference value between the first and second voltages V1 and V2.

$${V}_{out} = {V}_{2} - {V}_{1} = ( \frac{R}{{R}_{c 2} + R} - \frac{R}{{R}_{c 1} + R} ) \times {V}_{cc} ( \frac{R}{{R}_{2} + R} \times {V}_{egg 2} - \frac{R}{{R}_{1} + R} \times {V}_{egg 1} ) \qquad Equation 6$$

If the first and second brainwave sensor units 110 and 120 firmly contact the living body 10, since R_{c1} and R_{c2} are very small, Vₒᵤₜ is approximately given as shown in Equation 7.

Vₒᵤₜ=V_{eeg2}−V_{eeg1} Equation 7

The output value Vₒᵤₜ differential-amplified by the differential amplifier 250 may be postprocessed by using an adaptive filter (not shown) and may be analyzed.

To measure the brainwave signals, the first and second brainwave sensor units 110 and 120 are placed to physically contact the living body 10. However, if a user moves while the brainwave signals are being measured, motion may slightly occur between the first and second brainwave sensor units 110 and 120 and the living body 10. Due to the motion between the first and second brainwave sensor units 110 and 120 and the living body 10, the strength of contact may be changed, the strength of contact may be maintained but contact surfaces may slide aside, or the strength of contact may be changed and the contact surfaces may slide aside. As described above, if motion occurs on the contact surfaces between the first and second brainwave sensor units 110 and 120 and the living body 10, impedance variations occur. The impedance variations serve as noise (motion artifact) to brainwave signals collected by a biosignal measurement apparatus and thus the measured signals may have waveform distortion.

Since the first and second voltages V1 and V2 are measurable from the first and second voltage dividers (operational amplifiers) 210 and 220, respectively, R_{c1} and R_{c2} may be calculated by using Equations 2 and 4. If the distance between the first and second contact electrodes 111 and 112 of the first brainwave sensor unit 110 and the distance between the third and fourth contact electrodes 121 and 122 of the second brainwave sensor unit 120 are both denoted by d and the value of d is sufficiently reduced, the first skin resistance Rₛ₁ may be considered to be the same as the second skin resistance Rₛ₂ of the second brainwave sensor unit 120. Furthermore, when three or more brainwave sensor units are used, if the distances between contact electrodes of all brainwave sensor units are sufficiently reduced to the same value, a skin resistance thereof may be regarded as a resistance having a constant value. Therefore, the first contact resistance R₁ of the first brainwave sensor unit 110 and the third contact resistance R₂ of the second brainwave sensor unit 120 may be calculated by using Equations 3 and 5, respectively.

The first or third contact resistance R₁ or R₂ has a value which varies in real time in accordance with motion of the user. Therefore, by calculating the first or third contact resistance R₁ or R₂, the size and occurrence of motion artifact which varies as time passes may be measured and compensated in real time and thus signal analysis may be very easily performed.

As shown in Equations 2 to 5, since the first and second voltages V1 and V2 do not have variable related to each other, motion artifact may be independently estimated without influencing each other. That is, when three or more brainwave sensor units are used, since the brainwave sensor units independently measure motion artifact, although motion artifact occurs in any one brainwave sensor unit, the other brainwave sensor units are not influenced. As such, the brainwave measurement apparatus may be easily used in daily life.

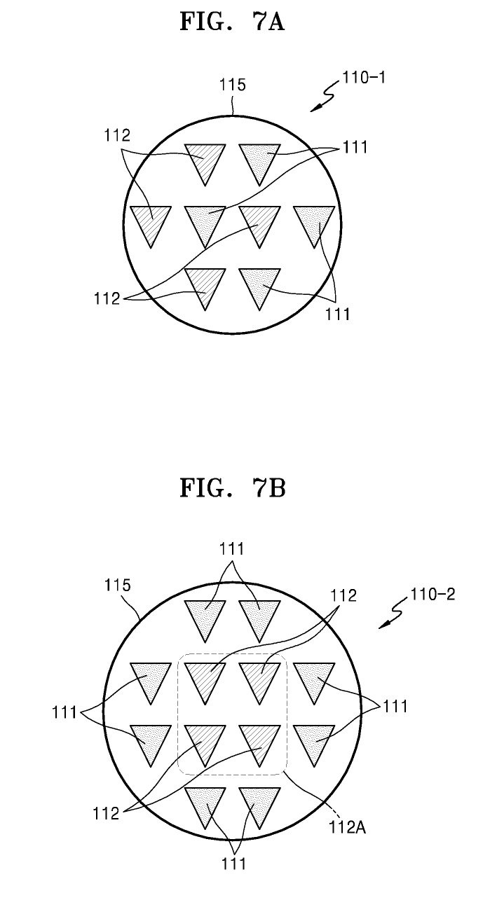

In the brainwave measurement apparatus according to the afore-described embodiments, each of the first and second brainwave sensor units 110 and 120 includes two contact electrodes, but is not limited thereto. FIG. 7A shows another example of contact electrode arrangement of a brainwave sensor unit 110-1. Referring to FIG. 7A, the brainwave sensor unit 110-1 may include four first contact electrodes 111 and four second contact electrodes 112. In this case, the first and second contact electrodes 111 and 112 may be provided in pairs located adjacent to each other. The four first contact electrodes 111 and the four second contact electrodes 112 may be uniformly distributed. The four first contact electrodes 111 may be electrically connected to each other and be connected to one signal line (e.g., the first signal line 113 of FIG. 2B). The four second contact electrodes 112 may be electrically connected to each other and be connected to one ground line (e.g., the first ground line 114 of FIG. 2B). The four first contact electrodes 111 are electrically separated from the four second contact electrodes 112. That is, the brainwave sensor unit 110-1 has a plurality of contact points physically contacting the living body 10 but may be interpreted as having two electrical contact points. The brainwave sensor unit 110-1 according to the current embodiment includes each of the first and second contact electrodes 111 and 112 by four, but is not limited thereto. For example, the brainwave sensor unit 110-1 may include each of the first and second contact electrodes 111 and 112 by two, three, five, or more.

FIG. 7B shows another example of contact electrode arrangement of a brainwave sensor unit 110-2. Referring to FIG. 7B, the brainwave sensor unit 110-2 may include eight first contact electrodes 111 provided along an outer side, and four second contact electrodes 112 located inside the eight first contact electrodes 111. The eight first contact electrodes 111 may be electrically connected to each other and be connected to one signal line (e.g., the first signal line 113 of FIG. 2B). The four second contact electrodes 112 may be electrically connected to each other and be connected to one ground line (e.g., the first ground line 114 of FIG. 2B). The eight first contact electrodes 111 are electrically separated from the four second contact electrodes 112. The brainwave sensor unit 110-2 according to the current embodiment includes the eight first contact electrodes 111 provided outside and the four second contact electrodes 112 provided inside, but is not limited thereto. For example, the number of the first contact electrodes 111 and the number of the second contact electrodes 112 may vary. In addition, the second contact electrodes 112 may be provided outside and the first contact electrodes 111 may be provided inside the second contact electrodes 112.

Since the number of the first contact electrodes 111 configured to measure brainwave signals is greater than the number of the second contact electrodes 112 to be grounded, a maximum number of the first contact electrodes 111 may be ensured within a limited space and thus the strength of the measured brainwave signals may be increased.

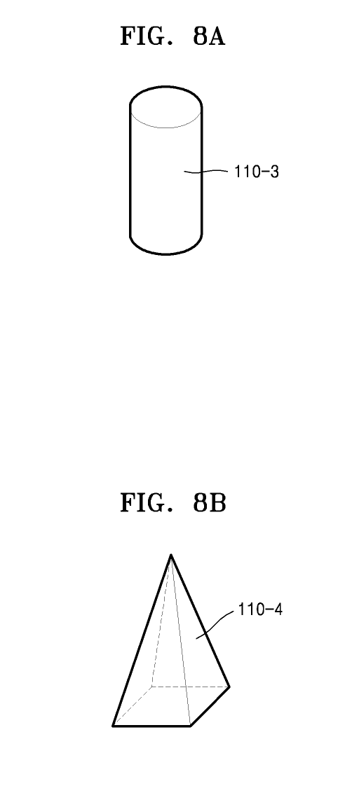

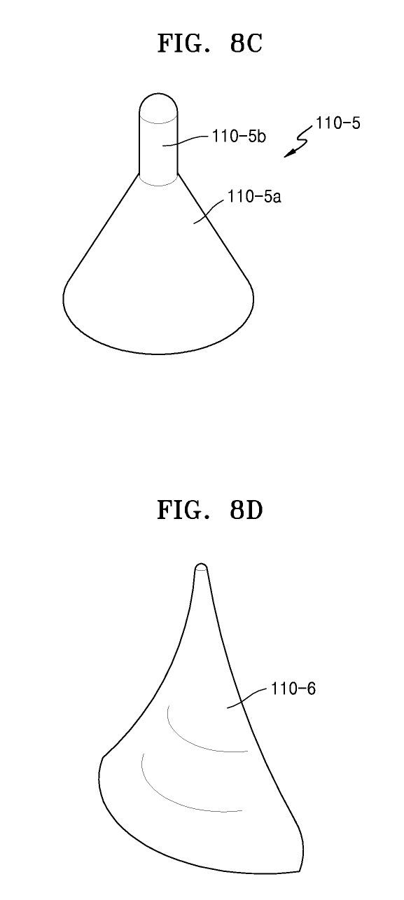

In the brainwave measurement apparatus according to the afore-described embodiments, each of the first and second brainwave sensor units 110 and 120 includes cone-shaped contact electrodes, but is not limited thereto. FIGS. 8A to 8D show examples of contact electrodes of a brainwave sensor unit. For example, as illustrated in FIG. 8A, contact electrodes 110-3 of the brainwave sensor unit may have a cylinder shape. As another example, as illustrated in FIG. 8B, contact electrodes 110-4 of the brainwave sensor unit may have a quadrangular pyramid shape. Otherwise, as illustrated in FIG. 8c, contact electrodes 110-5 of the brainwave sensor unit may have a funnel shape including a tapered part 110-5a having a tapered shape gradually reduced in diameter toward one end thereof, and a protruding part 110-5b provided on the end of the tapered part 110-5a. As another example, as illustrated in FIG. 8d, contact electrodes 110-6 of the brainwave sensor unit may have a curved funnel shape gradually reduced in diameter toward one end thereof. The contact electrodes may also have various pyramid shapes (e.g., a triangular pyramid shape and a pentagonal pyramid shape), an elliptical cone, polygonal prism shapes (e.g., a rectangular prism shape). Other known electrode structures may be employed for the contact electrodes.

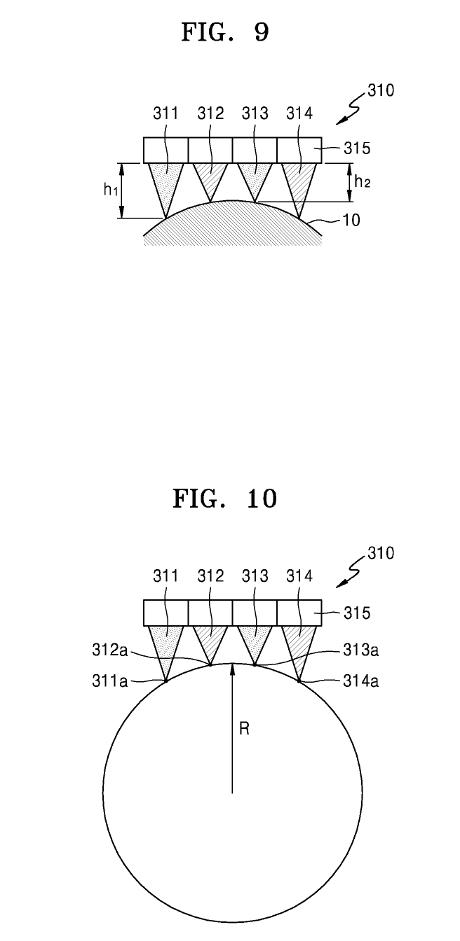

In the brainwave measurement apparatus according to the afore-described embodiments, each of the first and second brainwave sensor units 110 and 120 includes same-sized contact electrodes, but is not limited thereto. FIG. 9 is a side cross-sectional view of a brainwave sensor unit 310 according to another embodiment.

Referring to FIG. 9, the brainwave sensor unit 310 includes first to fourth contact electrodes 311, 312, 313, and 314, and a supporter 315 configured support the first to fourth contact electrodes 311, 312, 313, and 314. The first to fourth contact electrodes 311, 312, 313, and 314 are made of the same material. In addition, the first to fourth contact electrodes 311, 312, 313, and 314 may have the same shape, but some or all of the first to fourth contact electrodes 311, 312, 313, and 314 may have different heights. That is, heights h1 and h2 of the first to fourth contact electrodes 311, 312, 313, and 314 may be set based on the shape of the living body 10, e.g., a head shape. For example, the height h1 of the first and fourth contact electrodes 311 and 314 may be set to be greater than the height h2 of the second and third contact electrodes 312 and 313. The shape of the living body 10, e.g., the head shape, has a different average size depending on gender, age, etc. Therefore, representative sizes of the head shape may be classified based on gender, age, etc., and the brainwave sensor unit 310 having the heights h1 and h2 optimized for each size may be provided. Alternatively, the brainwave sensor unit 310 having the heights h1 and h2 optimized for the head shape of a specific user may be provided. As described above, by setting the heights h1 and h2 of the first to fourth contact electrodes 311, 312, 313, and 314 based on the shape of the living body 10, contact areas between the first to fourth contact electrodes 311, 312, 313, and 314 and the living body 10 may be increased and thus noise may be reduced. In addition, by uniformizing the contact areas between the first to fourth contact electrodes 311, 312, 313, and 314 and the living body 10, motion artifact may be more effectively reduced.

Some of the first to fourth contact electrodes 311, 312, 313, and 314 obtain brainwave signals from the living body 10, and the others are grounded. For example, the first and third contact electrodes 311 and 313 may obtain brainwave signals to sum and transmit the brainwave signals to the signal processor 200 (see FIG. 1), and the second and fourth contact electrodes 312 and 314 may be grounded.

Furthermore, as illustrated in FIG. 10, the heights of the first to fourth contact electrodes 311, 312, 313, and 314 may be set in such a manner that ends 311a, 312a, 313a, and 314a of the first to fourth contact electrodes 311, 312, 313, and 314 circumscribe a circle having a radius R. The head of a user may be approximated to a hemisphere shape. Therefore, head sizes may be classified into radii R based on gender, age, etc., and the brainwave sensor unit 310 having the first to fourth contact electrodes 311, 312, 313, and 314 which circumscribe a circle having each radius R may be provided.

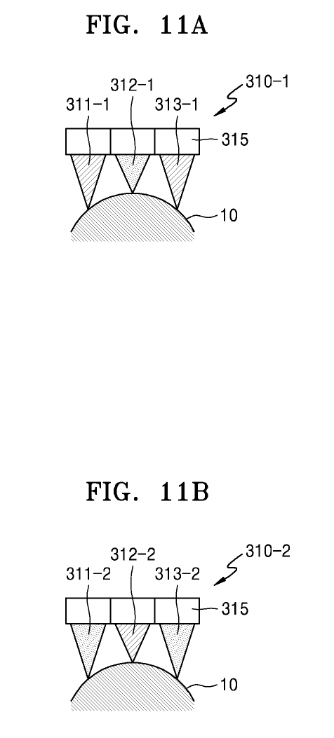

FIGS. 11A and 11B show modified examples of the brainwave sensor unit 310 of FIG. 9. For example, as illustrated in FIG. 11A, a brainwave sensor unit 310-1 may include two contact electrodes 311-1 and 313-1 to be grounded, and one contact electrode 312-1 located between the two contact electrodes 311-1 and 313-1 and configured to obtain a brainwave signal. In this case, the height of the contact electrode 312-1 configured to obtain the brainwave signal is set to be less than that of the two contact electrodes 311-1 and 313-1 to be grounded. Alternatively, as illustrated in FIG. 11B, a brainwave sensor unit 310-2 may include two contact electrodes 311-2 and 313-2 configured to obtain brainwave signals, and one contact electrode 312-2 located between the two contact electrodes 311-2 and 313-2 and to be grounded. In this case, the height of the two contact electrodes 311-2 and 313-2 configured to obtain the brainwave signals is set to be greater than that of the contact electrode 312-2 to be grounded.

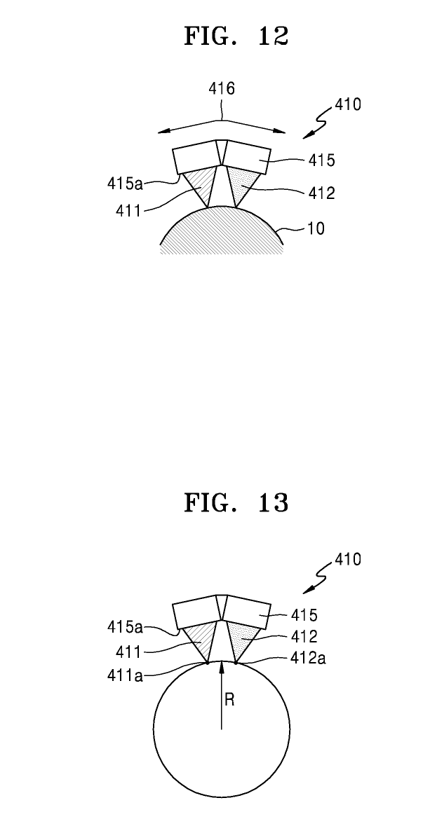

In the brainwave measurement apparatuses 310, 310-1, and 310-2 described above in relation to FIGS. 9, 10, 11A, and 11B, some or all of contact electrodes of each of the first and second brainwave sensor units 110 and 120 have different heights, but are not limited thereto. FIG. 12 is a side cross-sectional view of a brainwave sensor unit 410 according to still another embodiment.

Referring to FIG. 12, in the brainwave sensor unit 410 according to the current embodiment, first and second contact electrodes 411 and 412 have the same height (i.e., size), but a support surface 415a of a supporter 415 is bent. The first contact electrode 411 obtains a brainwave signal from the living body 10, and the second contact electrode 412 is grounded. Flexion 416 of the support surface 415a of the supporter 415 may be set in such a manner that the first and second contact electrodes 411 and 412 uniformly contact the living body 10. Representative sizes of a head shape may be classified based on gender, age, etc. as described above, and the brainwave sensor unit 410 having the flexion 416 optimized for each size may be provided. Alternatively, the brainwave sensor unit 410 having the flexion 416 optimized for the head shape of a specific user may be provided. As described above, by setting the flexion 416 of the supporter 415 based on the shape of the living body 10, contact areas between the first and second contact electrodes 411 and 412 and the living body 10 may be increased and thus noise may be reduced. In addition, by uniformizing the contact areas between the first and second contact electrodes 411 and 412 and the living body 10, motion artifact may be more effectively reduced.

Furthermore, as illustrated in FIG. 13, the flexion 416 of the supporter 415 may be set in such a manner that ends 411a and 412a of the first and second contact electrodes 411 and 412 circumscribe a circle having each of radii R classified based on gender, age, etc.

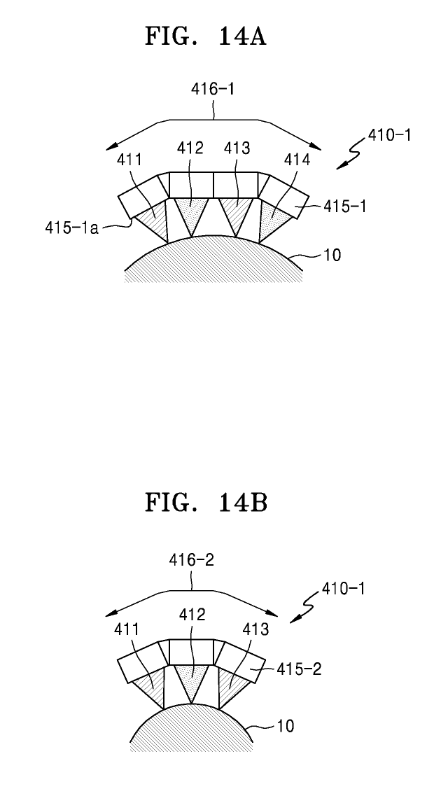

FIGS. 14A and 14B show modified examples of the brainwave sensor unit 410 of FIG. 12. For example, as illustrated in FIG. 14A, a brainwave sensor unit 410-1 includes first to fourth contact electrodes 411, 412, 413, and 414, and a supporter 415-1 configured to support the first to fourth contact electrodes 411, 412, 413, and 414. The first to fourth contact electrodes 411, 412, 413, and 414 may be made of the same material and may have the same shape and the same size. Some of the first to fourth contact electrodes 411, 412, 413, and 414 obtain brainwave signals from the living body 10, and the others are grounded. For example, the first and third contact electrodes 411 and 413 may obtain brainwave signals to sum and transmit the brainwave signals to the signal processor 200 (see FIG. 1), and the second and fourth contact electrodes 412 and 414 may be grounded. Flexion 416-1 of a support surface 415-1a of the supporter 415-1 may be set in such a manner that the first to fourth contact electrodes 411, 412, 413, and 414 uniformly contact the living body 10. For example, a part of the supporter 415-1 corresponding to the second and third contact electrodes 412 and 413 may be flat, and parts of the supporter 415-1 corresponding to the first and fourth contact electrodes 411 and 414 located at two sides of the second and third contact electrodes 412 and 413 may be bent. As another example, as illustrated in FIG. 14B, a brainwave sensor unit 410-2 may include two contact electrodes 411 and 413 configured to obtain brainwave signals, and one contact electrode 412 located between the two contact electrodes 411 and 413 and to be grounded. The first to third contact electrodes 411, 412, and 413 may be made of the same material and may have the same shape and the same size, but flexion 416-2 of a support surface 415-2a of a supporter 415-2 may be set in such a manner that the first to third contact electrodes 411, 412, and 413 uniformly contact the living body 10.

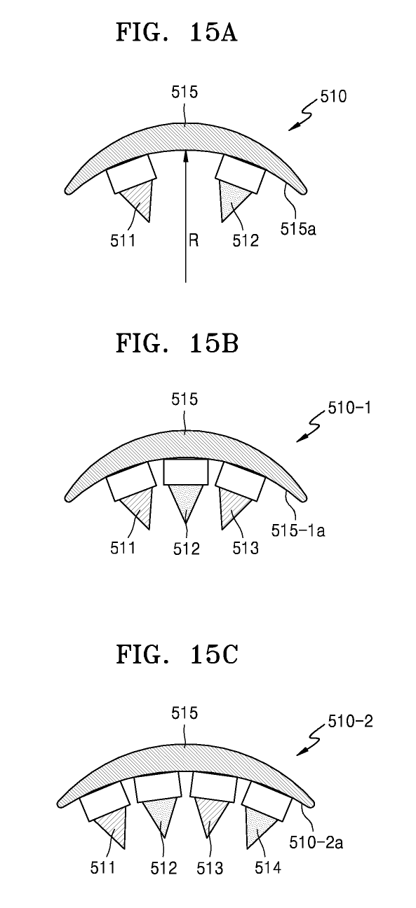

In the embodiments described above in relation to FIGS. 12, 13, 14A, and 14B, the supporters 415, 415-1, and 415-2 are bent, but are not limited thereto. FIGS. 15A to 15C show other modified examples of the brainwave sensor unit 410 of FIG. 13. For example, as illustrated in FIG. 15A, in a brainwave sensor unit 510, first and second contact electrodes 511 and 512 have the same height (i.e., size), but a support surface 515a of a supporter 515 is curved. The first contact electrode 511 obtains a brainwave signal from the living body 10, and the second contact electrode 512 is grounded. The support surface 515a of the supporter 515 is curved in such a manner that the first and second contact electrodes 511 and 512 uniformly contact the living body 10. Since the head of a user may be approximated to a hemisphere shape as described above, head sizes may be classified into radii R based on gender, age, etc., and the brainwave sensor unit 510 corresponding to each radius R may be provided. In other words, the support surface 515a of the supporter 515 may be set to circumscribe a circle having a radius R based on the head size. That is, a curvature radius of the support surface 515a of the supporter 515 may be set to be the radius R based on the head size. As such, contact areas between the first and second contact electrodes 511 and 512 and the living body 10 may be increased and thus noise may be reduced. In addition, by uniformizing the contact areas between the first and second contact electrodes 511 and 512 and the living body 10, motion artifact may be more effectively reduced.

As another modified example, as illustrated in FIG. 15B, a brainwave sensor unit 510-1 may include three contact electrodes 511, 512, and 513, and the support surface 515a of the supporter 515 may correspond to a head size of a user. Likewise, as illustrated in FIG. 15C, a brainwave sensor unit 510-2 may include four contact electrodes 511, 512, 513, and 514, and the support surface 515a of the supporter 515 may correspond to a head size of a user. The number of contact electrodes provided on the supporter 515 does not limit the current embodiment, and five or more contact electrodes may be provided.

The brainwave sensor unit according to the afore-described embodiments is connected to the signal processor in a wired manner, but is not limited thereto. The brainwave sensor unit may include a wireless communication module, and may transmit an obtained brainwave signal to the signal processor in a wireless manner.

FIG. 16 is a block diagram of a brainwave measurement apparatus 1100 according to another embodiment.

Referring to FIG. 16, the brainwave measurement apparatus 1100 includes a sensor 1110 and a circuit 1120. The sensor 1110 includes first and second brainwave sensor units 1111 and 1112 configured to measure brainwave signals from the living body 10. The first and second brainwave sensor units 1111 and 1112 have the same structure as the brainwave sensor unit according to the afore-described embodiments.

The circuit 1120 may include a signal processor 1121, a controller 1122, a communication unit 1123, a memory 1124, and an output unit 1125. Signals generated by the signal processor 1121, the controller 1122, the communication unit 1123, the memory 1124, and the output unit 1125 may be transmitted through a data bus 1126.

The signal processor 1121 generates a meaningful brainwave signal by using first and second brainwave signals obtained by the sensor 1110. The signal processor 1121 may differential-amplify the first and second brainwave signals obtained by the sensor 1110, and remove motion artifact mixed in the first and second brainwave signals. Furthermore, the signal processor 1121 may process the differential-amplified brainwave signal by dividing the same into α waves, β waves, γ waves, etc. per frequencies, or may perform other types of postprocessing. The signal processor 1121 may include the first and second voltage dividers 210 and 220 and the differential amplifier 250 as described above in relation to FIG. 5.

The controller 1122 may determine a state of a user based on the brainwave signal processed by the signal processor 1121. For example, the controller 1122 may determine whether the user is in an emergency situation, by analyzing the brainwave signal processed by the signal processor 1121, based on an algorithm of a preset brainwave model. In some cases, a process of additionally processing the brainwave signal or determining the state of the user based on the brainwave signal may be performed by an external device (e.g., a mobile device 1200 of FIG. 17) which communicates with the brainwave measurement apparatus 1100 in a wired or wireless manner, thereby reducing the load of the controller 1122.

Furthermore, the controller 1122 controls various functions of the brainwave measurement apparatus 1100. For example, the controller 1122 may control the sensor 1110, the communication unit 1223, the output unit 1225, the memory 1124, etc. by executing programs stored in the memory 1124. For example, when the user is in an emergency situation, the controller 1122 may control the communication unit 1123 to transmit information about the emergency situation of the user to the external device, or control the output unit 1125 to output the information about the emergency situation. Otherwise, the controller 1122 may control a speaker (not shown) or a vibration module (not shown) to notify the user of the emergency situation.

The communication unit 1123 includes at least one of a wired communication module and a wireless communication module. The wireless communication module may include, for example, a short-range communication module or a mobile communication module. The short-range communication module refers to a module for communication within a predetermined short range. For example, short-range communication technology may include Wireless Local Area Network (WLAN), Wi-Fi, Bluetooth, ZigBee, Wi-Fi Direct (WFD), Ultra Wideband (UWB), Infrared Data Association (IrDA), Bluetooth Low Energy (BLE), Near Field Communication (NFC), etc., but is not limited thereto. The mobile communication module transmits and receives wireless signals to and from at least one of a base station, an external device, and a server in a mobile communication network. The wired communication module refers to a module for communication by using electrical signals or optical signals, and wired communication technology according to an embodiment may include twisted pair cable, coaxial cable, fiber optic cable, Ethernet cable, etc. The communication unit 1123 may transmit obtained brainwave information to the external device, or receive control signals or information required for signal processing, from the external device.

The memory 1124 may store raw data of first and second brainwave signals obtained by the sensor 1110 or store the brainwave signal processed by the signal processor 1121. In addition, the memory 1124 may store a program for controlling operation of the brainwave measurement apparatus 1100, brainwave model algorithms required to analyze brainwave signals, authentication information, etc. Furthermore, the memory 1124 may store user state information (e.g., a brainwave pattern corresponding to an emergency situation, and a brainwave pattern corresponding to a state which requires medication) such that the controller 1122 may determine the state of the user.

The output unit 1125 may output the brainwave signal obtained by the signal processor 1121, or the user state information determined based on the brainwave signal. The output unit 1125 may include at least one of a display for displaying information about a living body in the form of an image or text, a speaker for outputting voice or warning sound, a vibration unit for outputting a vibration signal, and a lamp for emitting light.

The circuit 1120 may include at least one of a battery and an energy harvest module for driving the sensor 1110 and the circuit 1120.

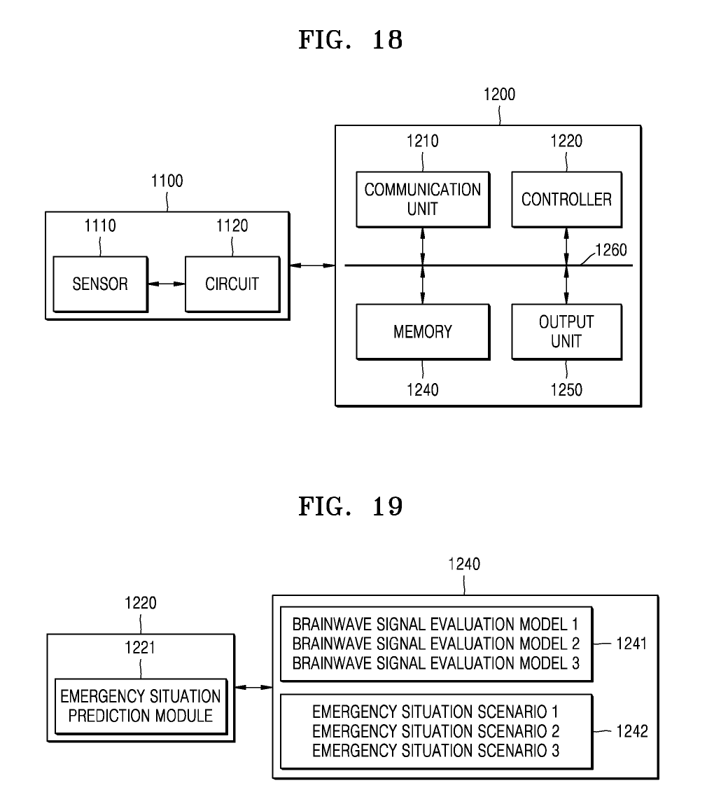

FIG. 17 is a block diagram of a brainwave measurement system according to an embodiment, FIG. 18 is a detailed block diagram of the brainwave measurement system of FIG. 17, and FIG. 19 shows an example of a controller 1220 and a memory 1240 of a mobile device 1200 in the brainwave measurement system of FIG. 18.

Referring to FIG. 17, the brainwave measurement system according to the current embodiment includes a brainwave measurement apparatus 1101 and the mobile device 1200 connected to the brainwave measurement apparatus 1101 in a wired or wireless manner.

The brainwave measurement apparatus 1101 includes the sensor 1110 configured to measure a brainwave signal of a user, and the circuit 1120 configured to process the brainwave signal measured by the sensor 1110. The brainwave measurement apparatus 1101 may be one of the brainwave measurement apparatuses according to the afore-described embodiments. The brainwave measurement apparatus 1101 may have a shape of an accessory worn by the user at ordinary times or a shape attached to the accessory, and thus may measure the brainwave signal of the user at any time. For example, a housing of the brainwave measurement apparatus 1101 may have any one shape among headphones, an earset, earphones, a hat, a hairband, glasses, a watch, a bracelet, a wristband, and an eye patch, or a shape attached thereto.

The mobile device 1200 may determine a state of the user based on the brainwave signal obtained by the brainwave measurement apparatus 1101. Referring to FIG. 18, the mobile device 1200 includes a communication unit 1210, the controller 1220, the memory 1240, and an output unit 1250. The mobile device 1200 may include a mobile phone, a smartphone, a tablet computer, a personal digital assistant (PDA), a laptop computer, etc., but is not limited thereto.

The communication unit 1210 communicates with the communication unit 1123 (see FIG. 16) provided in the circuit 1120 of the brainwave measurement apparatus 1101. The communication unit 1210 may include a wireless communication module, e.g., a WLAN, Wi-Fi, Bluetooth, ZigBee, WFD, UWB, IrDA, BLE, or NFC module, or a wired communication module. The communication unit 1210 receives the brainwave signal processed by the circuit 1120 of the brainwave measurement apparatus 1101, and transmits a control command to the circuit 1120 of the brainwave measurement apparatus 1101.

The controller 1220 processes the brainwave signal received from the circuit 1120, into meaningful biosignal data. The controller 1220 may include an emergency situation prediction module 1221 as illustrated in FIG. 19. The emergency situation prediction module 1221 predicts an emergency situation of the user who is wearing the brainwave measurement apparatus 1101, based on the biosignal data. The emergency situation prediction module 1221 may be implemented as software or hardware. When the emergency situation prediction module 1221 is implemented as software, the emergency situation prediction module 1221 may be stored in the memory 1240 and may be executed by the controller 1220 as necessary. The controller 1220 controls elements of the mobile device 1200, e.g., the communication unit 1210, the memory 1240, and the output unit 1250. The memory 1240 stores information related to processing of the brainwave signal. For example, the memory 1240 may include brainwave signal evaluation models 1241 required when the controller 1220 processes the brainwave information into the meaningful biosignal data. In addition, the memory 1240 may include emergency situation scenarios 1242 to be processed by the controller 1220 when the controller 1220 evaluates the brainwave signal and determines that a result of evaluation corresponds to an emergency situation. For example, the memory 1240 may include an address of a server of an emergency center, an address of a server of a hospital where the user usually goes, an address of a personal computer of the user, a phone number of a primary care doctor of the user, a phone number of a guardian of the user, etc. to contact in an emergency situation. The output unit 1250 may include a display for displaying the biosignal data or information related to the biosignal data. The output unit 1250 of the mobile device 1200 may further include known means capable of providing information to the user, e.g., a speaker and a vibration module.

The brainwave signal is always generated because the brain moves without a break, and diseases such as epilepsy, stroke, fainting, depression, dementia, and attention deficit hyperactivity disorder (ADHD) have unique brainwave features. Drowsiness and high stress also have unique brainwave features. Therefore, when the brainwave measurement apparatus 1101 measures the brainwave signal, the controller 1220 extracts brainwave features by processing the received brainwave signal. The brainwave signal evaluation models includes information about unique brainwave features of various diseases, and the emergency situation prediction module 1221 may determine an anomalous sign of the user by comparing the extracted brainwave features with the unique brainwave features of the diseases. Alternatively, the emergency situation prediction module 1221 may determine a risk level or an emergency level of a current state of the user by scoring a mild symptom, a severe symptom, etc. of each disease. The risk level means how risky the user is. The emergency level means how urgently the state of the user should be notified to another user (e.g., a doctor or a guardian) or how urgently the user should be treated. In many cases, the risk level and the emergency level may be used interchangeably. However, in some cases, the risk level may be high but the emergency level may be low, or vice versa. For example, drowsiness while driving a car has a very high risk level but a low emergency level. The risk level or the emergency level may be classified depending on the state of the user, a severity of a disease, or a degree of urgency. For example, a stroke suddenly occurs but has a pre-symptom such as facial paralysis, numbness in an arm or leg, or dysarthria in many cases. A mini stroke occurs temporarily and then resolves. When a severe stroke occurs, disturbance of consciousness and fainting may be caused and a function of the brain may be permanently disabled. Although some brain cells die quickly due to a stroke, other cells are damaged but may be saved through early medication. In addition, proper early treatment may prevent spread of the brain damage. Therefore, as will be described below with reference to Table 1, a risk level (or an emergency level) of a stroke based on a brainwave signal may be determined based on a severity of a stroke.

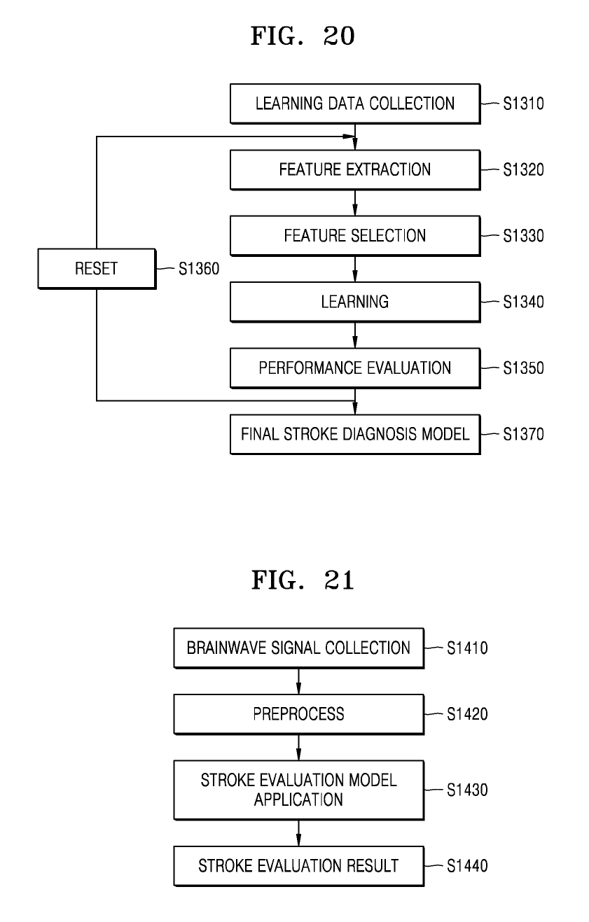

A process of determining a risk level or an emergency level of a stroke based on a brainwave signal by the emergency situation prediction module 1221 will now be described in detail with reference to FIGS. 20 to 22.

FIG. 20 shows a brainwave learning process for diagnosing a stroke.

Referring to FIG. 20, initially, learning data related to a stroke is collected (S1310). The learning data may include, for example, brainwave signals, gender information, age information, drinking information, and smoking information, and include both of data of normal people and data of stroke patients.

Then, features related to a stroke are extracted by processing the collected learning data (S1320). For example, one or a combination of various analysis functions such as frequency analysis functions (e.g., fast Fourier transform (FFT) and wavelet) and complexity analysis functions (e.g., multi-scale entropy (MSE) and correlation dimension) may be used.

Subsequently, an optimal feature having a high contribution to accuracy is selected from among the extracted features (S1330). For the selection, an algorithm such as Chi squared test, recursive feature elimination, least absolute shrinkage and selection operator (LASSO), elastic net, or ridge regression may be used.

Then, learning is performed by using a learning algorithm and a parameter (S1340). For the learning, a learning method such as multilayer perceptron, decision tree, support vector machine, or Bayesian network may be used.

Thereafter, performance evaluation is performed by using an evaluation method such as cross validation (S1350), and the learning algorithm and the parameter are reset (S1360) to repeat operations 1320, 1330, and 1340, thereby generating an optimal stroke diagnosis model (S1370).

The above stroke diagnosis model may be generated by a learning device and be implanted in the mobile device 1200. Otherwise, the stroke diagnosis model may be implemented by teaching the mobile device 1200. When the mobile device 1200 is taught, a neural network circuit may be provided in the mobile device 1200 in a hardware or software manner.

FIG. 21 shows a stroke evaluation process in the mobile device 1200.

Referring to FIG. 21, the mobile device 1200 collects diagnosis data (S1410). The diagnosis data includes a brainwave signal measured by the brainwave measurement apparatus 1100. A part of the diagnosis data may be input by a user or a third person (e.g., a medical personnel or a manufacturer). The diagnosis data may be data with the same condition as learning data.

Then, the controller 1220 of the mobile device 1200 extracts a feature by preprocessing the diagnosis data (S1420). The preprocessing may be performed in the same manner as learning.

Subsequently, the extracted feature is input to a stroke evaluation model (S1430), and it is predicted whether a stroke has occurred, by evaluating whether the feature is appropriate for the stroke evaluation model (S1440).

The prediction of whether a stroke has occurred may include determination of a risk level of a stroke.

Table 1 shows the National Institutes of Health Stroke Scale (NIHSS) as an example of the stroke evaluation model.

TABLE 1

| Model | NIHSS Score | Stroke risk level |
|---|---|---|
| Group 0 | 0\|1-42 | Whole test set |
| Group 1 | 0\|1 to 4 | Low severity |
| Group 2 | 0\|5 to 15 | Medium severity |
| Group 3 | 0\|16 to 20 | High severity |
| Group 4 | 0\|21 to 42 | Highest severity |

The NIHSS is a stroke scale of the National Institutes of Health of the United States, and groups of Table 1 are classified based on NIHSS scores. A group 0 evaluation model is a model for evaluating whether a stroke has occurred, and group 1 to group 4 evaluation models are models for evaluating severities of a stroke.

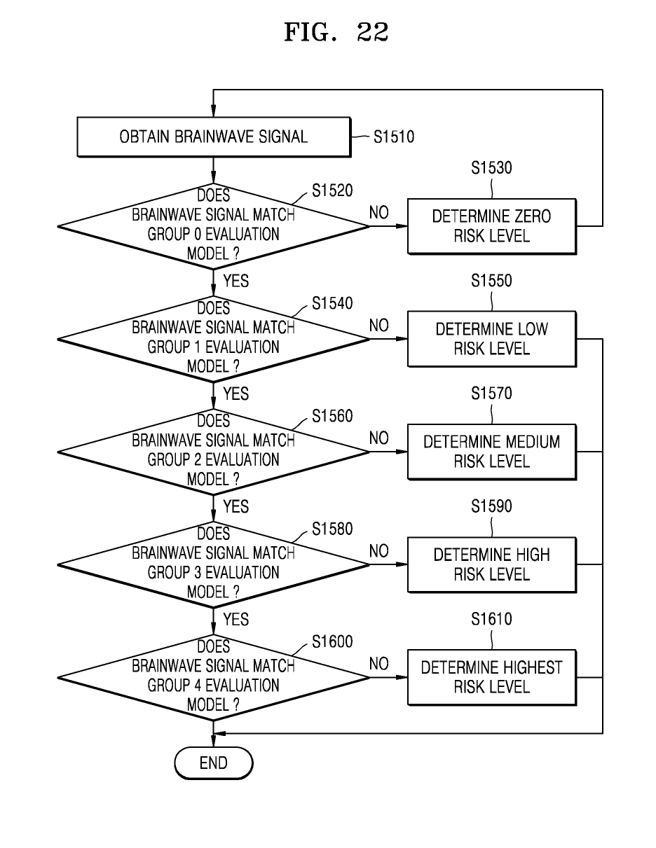

FIG. 22 is a flowchart of an example of a risk level determination process based on stroke evaluation by using the above group 0 to group 4 evaluation models.

Referring to FIG. 22, a brainwave signal is continuously obtained (S1510), and the obtained brainwave signal is applied to the group 0 evaluation model (S1520). If the obtained brainwave signal does not match the group 0 evaluation model, the process of obtaining the brainwave signal is repeated to continuously monitor whether a stroke has occurred. The fact that the obtained brainwave signal does not match the group 0 evaluation model means that a value calculated as a result of applying the obtained brainwave signal to the group 0 evaluation model corresponds to NHISS score 0. Since the NHISS score 0 means that a stroke has not occurred, if the obtained brainwave signal does not match the group 0 evaluation model, a stroke has not occurred and thus a zero stroke risk level may be determined (S1530).

If the obtained brainwave signal matches the group 0 evaluation model, a stroke severity evaluation process may start. In other words, if the value calculated as the result of applying the obtained brainwave signal to the group 0 evaluation model is equal to or greater than NHISS score 1, it may be determined that a stroke has occurred, and thus the stroke severity evaluation process (S1540 to S1610) may start.

Initially, the obtained brainwave signal is compared to the group 4 evaluation model (S1540). If a value calculated as a result of applying the obtained brainwave signal to the group 4 evaluation model is within a range of NIHSS scores 21 to 42, a highest stroke risk level is determined (S1550). If the value calculated as the result of applying the obtained brainwave signal to the group 4 evaluation model exceeds the range of NIHSS scores 21 to 42, a process of applying the obtained brainwave signal to the group 3 evaluation model (S1560) may start.

Then, the obtained brainwave signal is applied to the group 3 evaluation model (S1560). If a value calculated as a result of applying the obtained brainwave signal to the group 3 evaluation model is within a range of NIHSS scores 16 to 20, a high stroke risk level is determined (S1570). If the value calculated as the result of applying the obtained brainwave signal to the group 3 evaluation model exceeds the range of NIHSS scores 16 to 20, a process of applying the obtained brainwave signal to the group 2 evaluation model (S1580) may start.

Subsequently, the obtained brainwave signal is applied to the group 2 evaluation model (S1580). If a value calculated as a result of applying the obtained brainwave signal to the group 2 evaluation model is within a range of NIHSS scores 5 to 15, a medium stroke risk level is determined (S1590). If the value calculated as the result of applying the obtained brainwave signal to the group 2 evaluation model exceeds the range of NIHSS scores 5 to 15, a process of applying the obtained brainwave signal to the group 1 evaluation model (S1600) may start.

Then, the obtained brainwave signal is applied to the group 1 evaluation model (S1600). If a value calculated as a result of applying the obtained brainwave signal to the group 1 evaluation model is within a range of NIHSS scores 1 to 4, a low stroke risk level is determined (S1610). If the value calculated as the result of applying the obtained brainwave signal to the group 1 evaluation model exceeds the range of NIHSS scores 1 to 4, the process of obtaining the brainwave signal (S1510) may start again.

The risk level of a stroke may be evaluated by using a combination of different evaluation models. For example, if fast Fourier transform (FFT), multi-scale entropy (MSE), and correlation dimension are used, an evaluation model obtained by learning a result of FFT (FFT_MODEL), an evaluation model obtained by learning a result of MSE (MSE_MODEL), and an evaluation model obtained by learning a result of correlation dimension (Corel_MODEL) may be learned and performances thereof may be evaluated through cross validation. An evaluation result of each evaluation model (TrainResult) is calculated to a value between 0 and 1. A weight of the evaluation result of each evaluation model may be calculated by using Equations 8 to 10.

$$\begin{matrix}{Weight}_{FFTMODEL} = \frac{{TrainResult}_{FFTMODEL}}{( \begin{matrix}{TrainResult}_{FFTMODEL} + \\ {TrainResult}_{MSEMODEL} + {TrainResult}_{CorelMODEL}\end{matrix} )} & Equation 8 \\ {Weight}_{MSEMODEL} = \frac{{TrainResult}_{MSEMODEL}}{( \begin{matrix}{TrainResult}_{FFTMODEL} + \\ {TrainResult}_{MSEMODEL} + {TrainResult}_{CorelMODEL}\end{matrix} )} & Equation 9 \\ {Weight}_{CorelMODEL} = \frac{{TrainResult}_{CorelMODEL}}{( \begin{matrix}{TrainResult}_{FFTMODEL} + \\ {TrainResult}_{MSEMODEL} + {TrainResult}_{CorelMODEL}\end{matrix} )} & Equation 10\end{matrix}$$

A final stroke evaluation result (PredictResult) may be obtained by using Equation 11.

PredictResult=PredictResult_{FFTMODEL}*Weight_{FFTMODEL}+PredictResult_{MSEMODEL}*Weight_{MSEMODEL}+PredictResult_{CorelMODEL}*Weight_{CorelMODEL} Equation 11

In Equation 11, the final stroke evaluation result is expressed as a value between 0 and 1, and indicates probability of a stroke.

Table 2 shows the probability of a stroke based on the value of the final stroke evaluation result (PredictResult).

TABLE 2

| PredictResult (x) | Probability of stroke |
|---|---|
| 0 ≤ x < 0.3 | Normal |
| 0.3 ≤ x < 0.7 | Suspicion of stroke |
| 0.7 ≤ x ≤ 1 | High probability of stroke |

As described above, the emergency situation prediction module 1221 may determine a risk level of a stroke based on the brainwave signal received from the brainwave measurement apparatus 1100. If the emergency situation prediction module 1221 determines that a current state of the user corresponds to an emergency situation, the controller 1220 may perform a process based on emergency situation scenarios stored in the memory 1240.

For example, the risk level may be divided into a first risk level, and a second risk level higher than the first risk level. In this case, the first risk level may correspond to a non-emergency situation in which the user may recognize a risk and act properly, and the second risk level may correspond to an emergency situation in which a risk of the user should be urgently notified to a hospital or a guardian. For example, in the stroke evaluation model based on Table 1, group 1 may be regarded as the first risk level, and groups 2 to 4 may be regarded as the second risk level. In the stroke evaluation model based on Table 2, a stroke evaluation result of 0.3 to 0.7 may be regarded as the first risk level, and a stroke evaluation result of 0.7 to 1 may be regarded as the second risk level. If the emergency situation prediction module 1221 determines an early stage of a stroke, this may be regarded as the first risk level and thus the controller 1220 may issue an alert through the output unit 1250 of the mobile device 1200. As the alert, for example, a warning message or indication for notifying the user of an early stage of a stroke may be displayed on the output unit 1250, and a message for advising the user to go to a hospital for a checkup soon may be further displayed. When the mobile device 1200 includes a speaker or a vibration module, the alert may be issued by using the speaker or the vibration module. If the emergency situation prediction module 1221 determines a severe stroke, this may be regarded as the second risk level and thus the controller 1220 may provide information about an emergency situation of the user to a pre-registered emergency center, a hospital, or a guardian through the communication unit 1210. The information about the emergency situation may include identification information of the user, the brainwave signal obtained by the brainwave measurement apparatus 1100, and the stroke severity information of the user determined by the mobile device 1200. Furthermore, when the mobile device 1200 includes a location tracking device such as a global positioning system (GPS), the information about the emergency situation may include location information of the mobile device 1200 (i.e., location information of the user). Otherwise, the information about the emergency situation may include a treatment history of the user or contact information of a preset hospital or a primary care doctor.

The risk level may be further divided. For example, group 4 in the stroke evaluation model based on Table 1 corresponds to the highest severity of a stroke, and may be regarded as a highest risk level which requires very urgent treatment. Therefore, when the emergency situation prediction module 1221 determines the highest risk level of a stroke, the controller 1220 may output emergency sound at the highest volume through a speaker (not shown) embedded in the mobile device 1200, or notify an adjacent emergency worker, a doctor, or the like through a pre-registered emergency center or a server of a hospital to urgently process the emergency situation of the user. When the emergency situation prediction module 1221 determines the highest risk level of a stroke, the controller 1220 may request a mobile carrier to transmit a message for notifying the emergency situation and asking for help, to a mobile device located adjacent to the user and capable of communication.

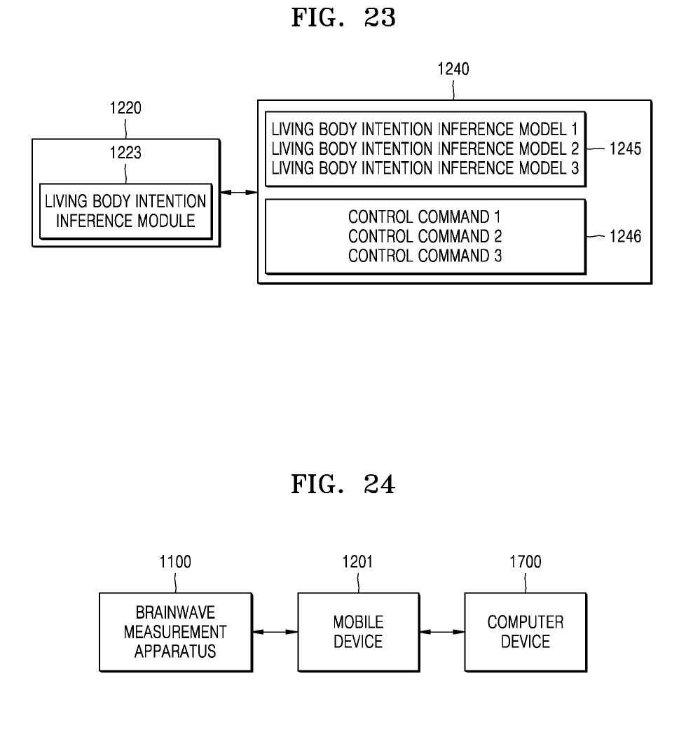

FIG. 23 is a block diagram of the controller 1220 and the memory 1240 of a mobile device 1201 according to another embodiment. Referring to FIG. 23, the controller 1220 includes a living body intention inference module 1223. The living body intention inference module 1223 infers thinking, i.e., an intention, of the user who wears the brainwave measurement apparatus 1101, from the processed brainwave information. The memory 1240 includes living body intention inference models 1245, and stores a set of control commands 1246 estimated by the living body intention inference models 1245. The living body intention inference models 1245 are obtained by modeling correlations between brainwave patterns and living body intentions. For example, when the brainwave measurement apparatus 1101 measures the brainwave signal, the received brainwave information may be analyzed per frequency components and be divided into α waves, β waves, γ waves, etc. The α waves, β waves, γ waves, etc. of the brainwave signal are dominantly provided in a range of 1 to 20 Hz, and a dominant frequency band varies depending on activity of the brain. The α waves, β waves, γ waves, etc. of the brainwave signal are related to activity of the brain. For example, the α waves are mainly measured from the frontal and temporal lobes and dominantly emerge in a relaxed state of the brain. The β waves emerge with anxiety, tension, or concentration and are the most evident in the frontal lobe. If the above described frequency characteristics and the brainwave emergence locations are combined, an activated part of the brain may be predicted. Considering that the brain has specific functions per locations, information about activity of the brain may be obtained. The living body intention inference module 1223 matches the obtained brainwave signal to a living body intention inference model, and infers the intention of the user from the matched living body intention inference model. The controller 1220 (see FIG. 18) may generate a control command for the mobile device 1200 or another electronic device based on the intention of the user inferred by the living body intention inference module 1223. Elements other than the living body intention inference module 1223 and the memory 1240 are the same as those of the mobile device 1200 according to the afore-described embodiments.

Although one of the emergency situation prediction module 1221 (see FIG. 19) and the living body intention inference module 1223 according to the afore-described embodiments is included in the mobile device 1200, both may be included in the mobile device 1200. Furthermore, the mobile device 1200 may include a healthcare module, a medication control module, etc. optimized for the user based on the biosignal data processed by the controller 1220.

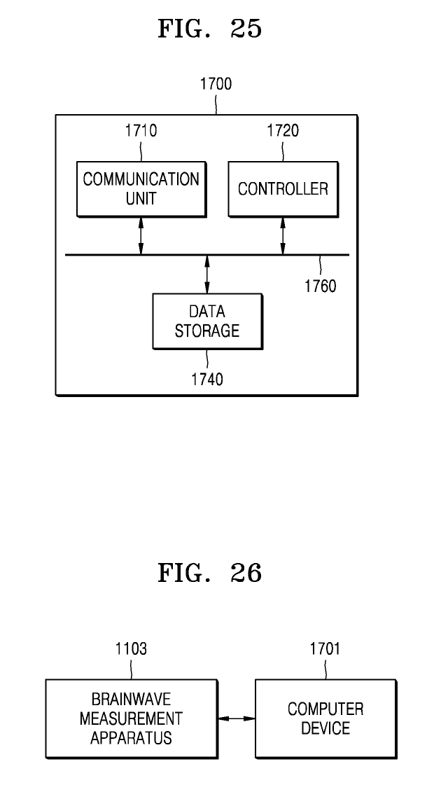

FIG. 24 is a block diagram of a brainwave measurement system according to another embodiment, and FIG. 25 is a detailed block diagram of a computer device 1700 in the brainwave measurement system of FIG. 24. Referring to FIGS. 24 and 25, the brainwave measurement system according to the current embodiment may include a brainwave measurement apparatus 1102, a mobile device 1201 connected to the brainwave measurement apparatus 1102 in a wired or wireless manner, and the computer device 1700 connected to the mobile device 1201 directly or via a network.

The computer device 1700 includes a communication unit 1710 configured to communicate with the mobile device 1201, a controller 1720 configured to process a brainwave signal received from the mobile device 1201 and to control various elements of the computer device 1700, and a data storage 1740 configured to store information related to processing of the brainwave signal. The communication unit 1710 may include a wireless communication module, e.g., a WLAN, Wi-Fi, Bluetooth, ZigBee, WFD, UWB, IrDA, BLE, or NFC module, or a wired communication module.

The computer device 1700 may perform at least some or all of brainwave signal processing processes. The mobile device 1201 transmits the brainwave information received from the brainwave measurement apparatus 1102, to the computer device 1700, and receives user state information analyzed by the computer device 1700. Although the mobile device 1200 according to the embodiments described above in relation to FIGS. 17 to 23 performs all brainwave signal processing processes such as stroke risk level analysis and user intention inference, according to the current embodiment, the mobile device 1201 performs some or no brainwave signal processing processes, and merely transmits the brainwave signal received from the brainwave measurement apparatus 1102, to the computer device 1700 or transmits the partially-processed brainwave signal to the computer device 1700. The data storage 1740 may include brainwave signal evaluation models used to evaluation the brainwave signal, and the controller 1720 may determine an emergency situation of a user or infer an intention of the user based on the brainwave signal evaluation models.

The computer device 1700 may be, for example, a server of a hospital, a server of an emergency center, or a personal computer of the user. The mobile device 1201 may transmit biosignal information of the user collected by the brainwave measurement apparatus 1102, to the computer device 1700, and the computer device 1700 may store the received biosignal information of the user and perform a subsequent procedure based on a scenario matched to a current state of the user.

As another example, the computer device 1700 may be an electronic device controllable by the mobile device 1201. In this case, the brainwave measurement system may be understood as a system in which the computer device 1700 is merely added to the brainwave measurement system described above in relation to FIGS. 17 to 23. That is, the brainwave signal processing processes such as stroke risk level analysis and user intention inference may be performed by the mobile device 1201, and the computer device 1700 may be an electronic device controlled by the mobile device 1201 (e.g., a home appliance such as a television, a lamp, a door lock system, or an air conditioner). For example, when the brainwave measurement apparatus 1102 measures the brainwave signal of the user as described above, the mobile device 1201 may infer the intention of the user and generate a control command for controlling the computer device 1700.

FIG. 26 is a block diagram of a brainwave measurement system according to still another embodiment. Referring to FIG. 26, the brainwave measurement system according to the current embodiment includes a brainwave measurement apparatus 1103 and a computer device 1701 connected to the brainwave measurement apparatus 1103 via a network. The brainwave measurement apparatus 1103 according to the current embodiment is directly connected to the computer device 1701 without the mobile device 1200 (see FIG. 17). The brainwave measurement apparatus 1103 may include the communication unit 1123 (see FIG. 16) connectable to a network and thus may be connected to the computer device 1700 via the network.

The brainwave signal processing processes described above in relation to FIGS. 18 to 23 may be performed by the brainwave measurement apparatus 1103. For example, the controller 1122 in the circuit 1120 (see FIG. 16) of the brainwave measurement apparatus 1103 may include an emergency situation prediction module or a living body intention estimation module, and the memory 1124 may store various brainwave signal evaluation models, emergency situation scenarios, etc. The controller 1122 determines a state of the user based on the brainwave signal processed by the signal processor 1121, and controls subsequent procedures based on the determined state of user. As another example, the brainwave signal processing processes may be performed by the computer device 1701 as in the embodiment described above in relation to FIGS. 24 and 25.

The computer device 1701 may be, for example, a server of a hospital or an emergency center, a desktop computer of the user, or a laptop computer. Furthermore, the computer device 1701 may be a home appliance connectable to a network. For example, when the user has a network environment having a wireless access point (WAP) and home appliances are connectable to the network, the brainwave measurement apparatus 1103 may be connected to the network through the wireless access point to control the home appliances.

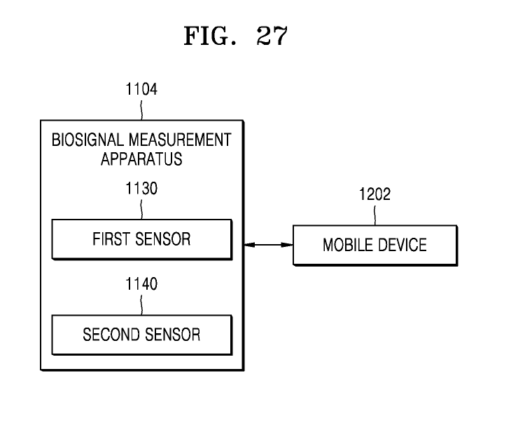

FIG. 27 is a block diagram of a brainwave measurement system according to still another embodiment. Referring to FIG. 27, the brainwave measurement system according to the current embodiment includes a biosignal measurement apparatus 1104 and a mobile device 1202. The biosignal measurement apparatus 1104 includes a first sensor 1130 and a second sensor 1140. The first sensor 1130 measures a brainwave signal, and may be a sensor of the brainwave measurement apparatus according to the afore-described embodiments. The second sensor 1140 includes a sensor electrode for measuring a biosignal other than the brainwave signal (e.g., electrocardiogram (ECG), electromyogram (EMG), electroneurogram (ENoG), or electrooculogram (EOG)), or another sensor for measuring a state of a user. For example, the second sensor 1140 may include at least one of a gyroscope sensor, an acceleration sensor, a global positioning system (GPS), a geomagnetic sensor, and an illumination sensor. The brainwave measurement system may be understood as a system in which the second sensor 1140 is added to the brainwave measurement apparatus described above in relation to FIGS. 17 to 23. The biosignal measurement apparatus 1104 obtains brainwave information from the brainwave signal of the user measured by the first sensor 1130, and collects additional information such as location information of the user, information indicating whether the user has fallen down, or information indicating whether the user is wandering the streets, by using the second sensor 1140. The biosignal measurement apparatus 1104 may transmit the brainwave information and the additional information to the mobile device 1202, and the mobile device 1202 may more accurately determine a current state of the user based on a combination of the brainwave information and the additional information. The second sensor 1140 may be included in the mobile device 1202 instead of the biosignal measurement apparatus 1104. The mobile device 1202 is an example of a biosignal processing device, and is not limited thereto. For example, the mobile device 1202 may be replaced with a computer device connected via a network. Alternatively, the biosignal measurement apparatus 1104 may process both of the brainwave signal and the additional information.

Examples to which the brainwave measurement system according to the afore-described embodiments is applied will now be described.

The brainwave measurement system according to the afore-described embodiments may be applied to the medical field. As described above, the brainwave measurement apparatus may be produced in various forms and be used in daily life. For example, the brainwave measurement apparatus may be produced in the form of a hat, glasses, a hairband, a hairpin, an eye patch, a patch, a pillow, a watch, a necklace, or a head-mounted display (HMD), or may be attached thereto. Therefore, if the user wears the brainwave measurement apparatus at ordinary times, the obtained biosignal information of the user may be used to prevent a disease or to diagnose a disease in an early stage in association with a hospital. For example, while the brainwave signal is being monitored, if an emergency situation is predicted or has occurred, the emergency situation may be notified to the user and, at the same time, the brainwave signal (indicating epilepsy, stroke, or the like) and the additional information such as the location information of the user may be transmitted to a medical institution or a health worker for diagnosis of a disease, emergency rescue, or treatment.

As another example, anxiety or panic in a case when a patient with dementia gets lost may be analyzed and, when the patient wanders the roads which have not been regularly used, state information and location information of the patient may be provided to a family member, a friend, a police, or the like to prevent disappearance.

As another example, neurofeedback (concentration training) customized for user characteristics (e.g., ADHD or age) may be provided.

As another example, a depression index may be generated based on a brainwave signal and be notified to a user or a medical worker, thereby enabling continuous monitoring. For example, when the brainwave signal indicates a high depression index, a message for recommending or instructing to take an antidepressant may be output to the user, thereby enabling medication control. Alternatively, a current treatment stage based on a medication may be notified by measuring a brainwave signal and thus the user may be encouraged to continuously receive treatment. The effect based on a history of taking the medication may be estimated by measuring the brainwave signal and thus the difference between before and after taking the medication may be notified. The effect of the medication may be notified to encourage the user to continuously receive treatment and thus the user may receive treatment for a long time. In addition, history information may be shared with a family member, a friend, or a medical worker and thus appropriate treatment may be provided.

As another example, a brainwave signal of a baby may be measured to recognize expression of an intention (e.g., hunger, sickness, or dislike). Since the brainwave signal is used, even when the baby does not cry, the intention of the baby may be recognized. An expression such as hunger, boredom, discomfort, drowsiness, stress, sleep status (e.g., sleeping or awaken), or emotion (e.g., like or dislike) may be recognized.

As another example, multimodal information may be extracted by using various form factors. For example, a body temperature, a heart rate, nodding, blinking, tossing and turning, etc. in addition to a brainwave signal may be measured at the same time, and thus accurate intention estimation and healthcare may be achieved.

As another example, the brainwave measurement system according to the afore-described embodiments may be applied to the safety and transportation fields. As described above, the brainwave measurement apparatus may be produced in various forms and thus may be produced in the form of a driver's seat, a hat, glasses, a hairband, a hairpin, an eye patch, a patch, or a pillow, or may be attached thereto. Therefore, the brainwave measurement apparatus may measure a brainwave signal of a user at any time. For example, when the user wears a brainwave measurement apparatus having a brainwave sensor on the head, a sleep status (e.g., drowsiness or concentration reduction) of safety and transportation workers may be diagnosed and an alarm may be output.

As another example, the brainwave measurement system according to the afore-described embodiments may be applied to the game field. For example, the brainwave measurement apparatus may be worn on the head to control a game or output an effect. As another example, a command may be transmitted by using a brainwave signal to control a virtual character (e.g., a brain computer interface (BCI)). As another example, a brainwave state (emotion) may be used to express an interactive game effect. For example, excitement of a user may be displayed by using the virtual character on a screen or may be reflected as an effect on the game.

As another example, the brainwave measurement system according to the afore-described embodiments may be applied to the home appliance field. As described above, the brainwave measurement apparatus may be produced in various forms and be used in daily life. For example, the brainwave measurement apparatus may be produced in the form of a hat, glasses, a hairband, a hairpin, an eye patch, a patch, a pillow, a watch, or a necklace, or may be attached thereto. For example, the brainwave measurement apparatus may be worn on the head of a user to operate (command) a smart home system and home appliances.

As another example, a state of a user may be monitored by using the brainwave measurement apparatus and thus an emergency situation of the user (e.g., fainting or encephalopathy) may be reported to an emergency center of a medical institution through a smart home system.

As another example, a state of a user may be monitored in real time in association with a Bluetooth device, a GPS, an acceleration sensor, a motion sensor, etc., and be transmitted to a smart home system (or home appliances).

As another example, a sleep status and a sleep depth may be detected by using a brainwave signal to transmit a command for operating smart home appliances. As such, illumination, temperature, humidity, etc. of a room when a user goes to bed, sleeps, or wakes may be properly controlled by measuring a sleep brainwave signal.

As another example, music played when a user goes to bed or wakes may be controlled by measuring a sleep brainwave signal.

As another example, by analyzing a brainwave signal of a user when the user watches multimedia content (e.g., TV), a highly interest/concentration period of the user may be determined to produce and then share highlight content with others through device connection or a cloud server.

As another example, a brainwave signal of a baby may be measured to recognize expression of an intention such as hunger, sickness, or dislike. Since the brainwave signal is used, even when the baby does not cry, the intention of the baby, such as hunger, boredom, discomfort, drowsiness, stress, sleep status (e.g., sleeping or awaken), or emotion (e.g., like or dislike), may be recognized.

In addition to a brainwave signal, multimodal information such as a body temperature, a heart rate, nodding, blinking, tossing and turning, etc. may be extracted by using various form factors, and thus accurate intention estimation and healthcare may be achieved.

As another example, the brainwave measurement system according to the afore-described embodiments may cooperate with a mobile device and be applied to the daily life field. The brainwave measurement apparatus may be worn on the head to construct a healthcare monitoring system for analyzing a brainwave signal of a user in real time. For example, the brainwave measurement apparatus may be worn on the head to manipulate a smartphone by using the brainwave signal.

As another example, when a brainwave signal is analyzed in real time, if a problem has occurred, an alarm may be immediately issued and a specific application may be executed or a text message may be provided by leaning brainwave signals of the user.

As another example, medication control may be enabled by using a brainwave signal. Since a brainwave signal before taking a medication differs from the brainwave signal after taking the medication, if the medication is not taken after a medication time, an alarm may be provided.

As another example, when a photo is taken, the photo may be stored together with emotion information. Thereafter, the photo is viewed together with the emotion information to achieve memory enhancement through retrospection. As such, a photo serendipity service may be provided.

As another example, a shutter of a camera may be pressed by using a brainwave signal. Furthermore, a face image of a user may be analyzed and captured by using a brainwave signal.

As another example, when a captured photo is stored, an emotion such as joy, depression, touching, sadness, anger, or love may be analyzed by using a brainwave signal.

As another example, a photo may be displayed on a home screen or a lock screen based on a pattern of using a mobile device. Additionally, a quiz about a location, time, or person related to a photo may be provided on the lock screen and the device may be unlocked if a correct answer is given, thereby proving memory enhancement training.

As another example, a high concentration period during a day may be notified to a user by using a brainwave signal such that the user may record a corresponding situation in a journal. For example, high concentration periods during a day may be automatically notified to the user to help the user to write memos about corresponding situations. The written memos may be automatically recorded in the journal.

As another example, a depression index may be measured by using a brainwave signal and an emoticon or photo appropriate for the depression index may be posted on a social networking site (SNS)/blog, thereby attracting interests of others.

As another example, a depression index may be analyzed based on a facial expression, a voice tone in a phone call, or a personal message posted on an SNS/blog, thereby providing an easy input function.

As another example, when an emotion is shared by using an SNS/blog, interests of others may be attracted in various user interface (UI)/user experience (UX) manners, e.g., an emoticon, a photo, and music.

As another example, a customized depression index may be determined in consideration of personality and conditions of an individual.

As another example, for online or offline shopping, preferences of a user may be determined by using a brainwave signal and a bookmark service may be provided based on the preferences.

As another example, the brainwave measurement system according to the afore-described embodiments may be applied to the education field. An apparatus having a brainwave sensor may be worn on the head to provide a customized education service may be proved based on educational achievements and interests of a user. In addition, personalized curriculums, levels, and teaching methods may be provided by analyzing concentration, excitement, and stress indices of the user. Furthermore, since comprehension and concentration of the user may be determined by using a brainwave signal, additional information (e.g., a hint) capable of encouraging the user in learning or a stimulation for increasing concentration of the user may be provided and the level of education may be controlled by changing content types based on comprehension of the user.

As another example, the brainwave measurement system according to the afore-described embodiments may be applied to the entertainment field. An apparatus having a brainwave sensor may be worn on the head to provide a service of recommending content based on a feeling of a user. In addition, by comprehensively analyzing a brainwave signal in terms of concentration, stress index, anxiety, etc., a wallpaper image may be changed, music may be automatically recommended, an application may be recommended, a restaurant may be recommended, a place may be recommended, a place to travel may be recommended, a shopping item may be recommended, the brightness of a screen may be adjusted, a font may be changed, or a frame (or a photo) may be displayed, based on the feeling of the user.

The device described herein may comprise a processor, a memory for storing program data and executing it, a permanent storage such as a disk drive, a communications port for handling communications with external devices, and user interface devices, including a touch panel, keys, buttons, etc. When software modules or algorithms are involved, these software modules may be stored as program instructions or computer-readable codes executable on the processor on a computer-readable medium. Examples of the computer-readable recording medium include magnetic storage media (e.g., ROM, floppy disks, hard disks, etc.), and optical recording media (e.g., CD-ROMs, or DVDs). The computer-readable recording medium can also be distributed over network coupled computer systems so that the computer-readable code is stored and executed in a distributed fashion. This media can be read by the computer, stored in the memory, and executed by the processor.

The present disclosure may be described in terms of functional block components and various processing steps. Such functional blocks may be realized by any number of hardware and/or software components configured to perform the specified functions. For example, the present disclosure may employ various integrated circuit components, e.g., memory elements, processing elements, logic elements, look-up tables, and the like, which may carry out a variety of functions under the control of one or more microprocessors or other control devices. Similarly, where the elements of the present disclosure are implemented using software programming or software elements the disclosure may be implemented with any programming or scripting language such as C, C++, Java, assembler, or the like, with the various algorithms being implemented with any combination of data structures, objects, processes, routines or other programming elements. Functional aspects may be implemented in algorithms that execute on one or more processors. Furthermore, the present disclosure could employ any number of conventional techniques for electronics configuration, signal processing and/or control, data processing and the like. The words “mechanism”, “element”, “means”, and “configuration” are used broadly and are not limited to mechanical or physical embodiments, but can include software routines in conjunction with processors, etc.

The particular implementations shown and described herein are illustrative examples of the disclosure and are not intended to otherwise limit the scope of the disclosure in any way. For the sake of brevity, conventional electronics, control systems, software development and other functional aspects of the systems may not be described in detail. Furthermore, the connecting lines, or connectors shown in the various figures presented are intended to represent functional relationships and/or physical or logical couplings between the various elements. It should be noted that many alternative or additional functional relationships, physical connections or logical connections may be present in a practical device.

The use of the terms “a” and “an” and “the” and similar referents in the context of describing the disclosure (especially in the context of the following claims) are to be construed to cover both the singular and the plural. Furthermore, recitation of ranges of values herein are merely intended to serve as a shorthand method of referring individually to each separate value falling within the range, unless otherwise indicated herein, and each separate value is incorporated into the specification as if it were individually recited herein.

While one or more embodiments have been described with reference to the figures, it will be understood by those of ordinary skill in the art that various changes in form and details may be made therein without departing from the spirit and scope as defined by the following claims.

1. A brainwave sensor unit comprising:
first and second contact electrodes having a tapered shape to contact a living body;
a signal line configured to transmit a brainwave signal obtained by the first contact electrode, to a signal processor;
a ground line configured to ground the second contact electrode; and
a supporter configured to separate and electrically insulate the first and second contact electrodes from each other.

2. The brainwave sensor unit of claim 1, wherein the first and second contact electrodes are made of a flexible material to protrude from a support surface of the supporter, and
wherein a distance between the first and second contact electrodes is determined based on a height and a base width of the first and second contact electrodes.

3. The brainwave sensor unit of claim 1, wherein a maximum distance between the first and second contact electrodes satisfies 80% of a correlation of the brainwave signal measured by the brainwave sensor unit with respect to a brainwave signal measured by a patch-type brainwave sensor.

4. The brainwave sensor unit of claim 1, wherein a distance between the first and second contact electrodes is between 0.5 mm and 5 mm.

5. The brainwave sensor unit of claim 1, wherein a number of the first contact electrodes is at least one, and
wherein the number of the first contact electrodes is equal to or greater than a number of the second contact electrodes.

6. The brainwave sensor unit of claim 1, wherein the first and second contact electrodes are provided in pairs located adjacent to each other.

7. The brainwave sensor unit of claim 1, wherein a support surface of the supporter comprises a first region and a second region, and
wherein a plurality of the first contact electrodes are provided on the first region and a plurality of the second contact electrodes are provided on the second region.

8. The brainwave sensor unit of claim 1, wherein a height of the first contact electrode is different from a height of the second contact electrode with respect to a support surface of the supporter.

9. The brainwave sensor unit of claim 1, wherein a height of the first contact electrode is equal to a height of the second contact electrode with respect to a support surface of the supporter, and
wherein the support surface of the supporter is bent or curved.

10. The brainwave sensor unit of claim 1, wherein a material of the first and second contact electrodes comprises one of conductive silicone, conductive rubber, and metal.

11. The brainwave sensor unit of claim 1, wherein the first and second contact electrodes have one of a cylinder shape, a cone shape, a quadrangular pyramid shape, a rectangular prism shape, a funnel shape, and a curved funnel shape.

12. A brainwave measurement apparatus comprising:
a first brainwave sensor unit comprising first and second contact electrodes having a tapered shape to contact a first location of a living body, a first signal line configured to transmit a first brainwave signal obtained by the first contact electrode, to a signal processor, a first ground line configured to ground the second contact electrode, and a first supporter configured to separate and electrically insulate the first and second contact electrodes from each other;
a second brainwave sensor unit comprising third and fourth contact electrodes having a tapered shape to contact a second location of the living body, a second signal line configured to transmit a second brainwave signal obtained by the third contact electrode, to the signal processor, a second ground line configured to ground the fourth contact electrode, and a second supporter configured to separate and electrically insulate the third and fourth contact electrodes from each other; and
the signal processor configured to process the first and second brainwave signals obtained by the first and second brainwave sensor units.

13. The brainwave measurement apparatus of claim 12, wherein the signal processor comprises:
a first voltage divider connected to the first signal line of the first brainwave sensor unit and a voltage source to output a first voltage signal voltage-divided from the first brainwave signal received from the first brainwave sensor unit, and the voltage source;
a second voltage divider connected to the second signal line of the second brainwave sensor unit and the voltage source to output a second voltage signal voltage-divided from the second brainwave signal received from the second brainwave sensor unit, and the voltage source; and
a differential amplifier configured to amplify a difference value between the first and second voltage signals.

14. The brainwave measurement apparatus of claim 13, wherein the signal processor extracts a first impedance between the first contact electrode of the first brainwave sensor unit and the living body from the first voltage signal output from the first voltage divider, extracts a second impedance between the third contact electrode of the second brainwave sensor unit and the living body from the second voltage signal output from the second voltage divider, and removes motion artifact from the first and second brainwave signals based on the first and second impedances.

15. The brainwave measurement apparatus of claim 12, further comprising:
a communication unit configured to communicate with an external device;
an output unit configured to output an alert; and
a controller configured to determine an emergency level of a user based on a brainwave signal processed by the signal processor, and to control the output unit to output an alert corresponding to the determined emergency level or control the communication unit to transmit information about the determined emergency level to the external device.

16. The brainwave measurement apparatus of claim 15, further comprising a memory configured to store a risk level evaluation model for evaluating a first risk level and a second risk level higher than the first risk level, based on the brainwave signal,
wherein the controller controls the output unit to output the alert if the emergency level of the user corresponds to the first risk level, or control the communication unit to transmit the information about the emergency level of the user to the external device if the emergency level of the user corresponds to the second risk level.

17. The brainwave measurement apparatus of claim 12, wherein a first distance between the first and second contact electrodes of the first brainwave sensor unit may be equal to a second distance between the third and fourth contact electrodes of the second brainwave sensor unit.
